(19) United States
(12) Patent Application Publication
(10) Pub. No.: US 2025/0304523 A1
(43) Pub. Date: Oct. 2, 2025

(54) COMBINED LACTATE AND KETONE BODY ESTERS FOR MEDICAL AND NUTRITIONAL USE

(71) Applicants: Aarhus Universitet, Aarhus C (DK); Region Midtjylland, Viborg (DK)

(72) Inventors: Thomas Bjørnskov Poulsen, Aarhus C (DK); Mogens Johannsen, Aarhus C (DK); Niels Møller, Aarhus C (DK); Alejandro José Mahía Moros, Aarhus C (DK); Jacob Marthinsen Seefeldt, Aarhus C (DK); Jakob Hansen, Aarhus C (DK); Bent Roni Ranghøj Nielsen, Aarhus C (DK)

(21) Appl. No.: 18/861,806

(22) PCT Filed: May 3, 2023
(86) PCT No.: PCT/EP2023/061662

§ 371 Date: Oct. 30, 2024

(30) Foreign Application Priority Data
May 4, 2022 (EP) 22171587.3

(51) Int. Cl. C07C 69/68; A61K 31/225

(52) U.S. Cl. CPC C07C 69/68; A61K 31/225

(57) ABSTRACT

The present invention relates to a Lactate/Ketone body ester for preservation of vital organ function and combat inflammation and cancer growth. In particular, the present invention relates to a Lactate/Ketone body ester with the beneficial properties of Lactate and beta-hydroxybutyrate (BHB) on vital organ function, inflammation and cancer growth but without the harmful sodium loads following administration of both Lactate and beta-hydroxybutyrate.

### TECHNICAL FIELD OF THE INVENTION

The present invention relates to Lactate/Ketone body esters for preservation of vital organ function and combat inflammation, cancer growth, sarcopenia, atherosclerosis, heart failure, obesity, diabetes, and metabolic syndrome. In particular, the present invention relates to Lactate/Ketone body esters with the beneficial properties of lactate or lactate and ketone bodies, such as beta-hydroxybutyrate (BHB), on vital organ function, inflammation, cancer growth, obesity, diabetes, and metabolic syndrome but without harmful ion loads, such as calcium potassium, magnesium and/or sodium loads following administration of both Lactate and beta-hydroxybutyrate salts.

### BACKGROUND OF THE INVENTION

Lactate (C₃H₆O₃) and ketone bodies, notably beta-hydroxybutyrate (BHB-C₄H₈O₃), are low molecular weight carbon fuel metabolites with many similarities: both are highly O₂ efficient agents produced and used by the human body as an energy source (1,2,8). They have protective properties during stress and act through similar, yet distinct, tissue receptors (1, 2). Lactate and ketone bodies contribute to the health-promoting effects of exercise, intermittent fasting, a ketogenic diet, and SGLT-2 inhibition in diabetes. In addition, preclinical studies strongly suggest that both compounds (alone or in combination) effectively counteract inflammation, cancer growth, protein loss and neurodegeneration (1, 2). We have recently found striking beneficial clinical effects of BHB in the heart, brain, skeletal muscle and bone marrow (increased Epo) in terms of increased blood flow and improved organ function (3-5). The possible beneficial effects of lactate are currently being investigated in human studies and Ringers lactate (Sodium lactate) is used routinely in the emergency room. Several pre-clinical studies suggest multiple beneficial effects of lactate (1,2,6-9). This also includes formation of lactate-derivatives, such as N-lactoyl-phenylalanine (Lac-Phe), that suppress appetite and thereby could be beneficial in patients that have a high BMI and/or diabetic conditions.

WO04/105742 teaches that compounds which reduce the level of free fatty acids circulating in the plasma of a subject may be used e.g. to treat muscle impairment or fatigue.

US 2011/0237666 A1 discloses 3-hydroxybutyl 3-hydroxybutyrate enantiomerically enriched with respect to (3R)-hydroxybutyl (3R)-hydroxybutyrate, as oral precursor of (3R)-hydroxybutyrate, for use in the treatment of a condition which is caused by, exacerbated by or associated with elevated plasma levels of free fatty acids in a human or animal subject; cognitive dysfunction, neurodegenerative diseases, for instance Alzheimer's disease, Parkinson's disease, Huntington's chorea, epilepsy; hypoxic states, for instance angina pectoris, extreme physical exertion, intermittent claudication, hypoxia, stroke and myocardial infarction; insulin resistant states, for instance infection, stress, obesity, diabetes, metabolic syndrome and heart failure; inflammatory states including infection and autoimmune disease and muscle impairment, fatigue and muscle fatigue. It is further disclosed that the compound reduces plasma levels of fatty acids and may be used for e.g. treating a condition, which is caused by, exacerbated by or associated with elevated plasma levels of free fatty acids in a human or animal subject.

WO 2012/131069 A1 describes short chain fatty acid derivatives such as propyl 3-hydroxypropionate and propyl propionate for use in the treatment of immunogenic disorders such as inflammatory diseases and viral infection (e.g. hepatitis).

PUCHALSKA PATRYCJA ET AL. (“Multi-dimensional Roles of Ketone Bodies in Fuel Metabolism, Signaling, and Therapeutics”, CELL METABOLISM, vol. 25, no. 2, 7 February 2017, pages 262-284) describes the multiple therapeutic implications of ketone bodies in e.g. inflammation and injury in multiple organ systems, heart failure, atherosclerosis, myocardial infarction, cancer, obesity, diabetes, NAFLD/NASH, diseases of the nervous system, oxidative stress.

Li et al (“An exercise-inducible metabolite that suppresses feeding and obesity”, NATURE, vol. 606, 15. June 2022, pages 785-790) N-lactoyl-phenylalanine (Lac-Phe), discloses that exercise stimulates the production of N-lactoyl-phenylalanine (Lac-Phe), a blood-borne signalling metabolite that suppresses feeding and obesity. There is, however, a compelling—and currently unmet—need for suitable preparations of lactate and ketone bodies such as BHB to avoid excessive ion loading such as sodium loading—and a horrible taste. These factors severely limit the amount of the metabolites that can be ingested, both with respect to scenarios involving daily ingestion as a food supplement and use in medicinally relevant settings.

Hence, a compound or a method for administration of such a compound to achieve the beneficial properties of lactate and BHB, while minimizing the harmful and unpleasant side effects would be of great importance.

### SUMMARY OF THE INVENTION

The present invention relates to the identification of compounds and compositions, which can release lactate or lactate and BHB in vivo, called lactate/ketone body esters. Thus, the compounds of the invention can be considered to be prodrugs and/or nutraceuticals. Surprisingly, the inventors have also devised a novel compound consisting of pyruvate and a BHB-precursor, which upon uptake and release thereof result in conversion into lactate and BHB. Thus without wishing to be bound by theory, the pyruvate-BHB compound (diPyKe) has the same effects as the lactate ketone body esters described herein.

The lactate/ketone body esters will have dual functions limiting e.g. inflammation of general relevance for treating or ameliorating aging-related diseases as well as they are high-energy metabolic substrates. The latter effect is both of relevance for medical use e.g. for improving muscle function or output and/or limiting muscle wasting in hospitalized patients or in the general aging-population. In addition the esters may be relevant as food or nutritional supplements in e.g. endurance or high-performance sports. The esters moreover have a range of further medical uses, e.g. to reduce appetite/food intake in patients suffering from obesity which could also serve to ameliorate diabetic conditions/symptoms. Lastly the compounds ability to reduce free fatty acids and increase Lac-Phe has relevance for treating or reducing obesity, diabetes, and metabolic syndrome.

Thus, an object of the present invention relates to providing a compound with the beneficial properties of lactate and BHB.

In particular, it is an object of the present invention to provide a compound, which solves the above-mentioned problems, without compromising the beneficial effects.

In the present context, unless otherwise specified the term “diLaKe” refers to compounds with the overall structure:

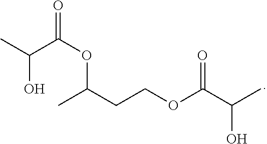 [SMILES: CC(CCOC(=O)C(C)O)OC(=O)C(C)O]

In the present context, unless otherwise specified the term “LaKe” refers to compounds with the overall structure:

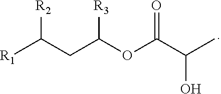

wherein R₁ is CH₃ or OH, R₂ is OH or H and R₃ is CH₃ or H, preferably wherein R₁, R₂ and R₃ are CH₃, OH and H respectively or R₁, R₂ and R₃ are OH, H and CH₃ respectively.

In the present context, unless otherwise specified the term “DiLa” refers to compounds with the overall structure:

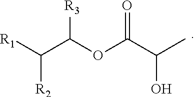

wherein R₁ is CH₃ or OH, R₂ is OH or H and R₃ is CH₃ or H, preferably wherein R₁, R₂ and R₃ are CH₃, OH and H respectively or R₁, R₂ and R₃ are OH, H and CH₃ respectively.

In the present context, unless otherwise specified the term “KeLa” refers to compounds with the overall structure:

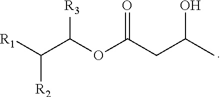

wherein R₁ is CH₃ or OH, R₂ is OH or H and R₃ is CH₃ or H, preferably wherein R₁, R₂ and R₃ are CH₃, OH and H respectively or R₁, R₂ and R₃ are OH, H and CH₃ respectively.

In the present context, unless otherwise specified the term “diPyKe” refers to a compound with the overall structure:

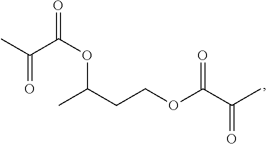 [SMILES: CC(=O)C(=O)OCCC(C)OC(=O)C(C)=O]

preferably with the stereochemistry according to the structure

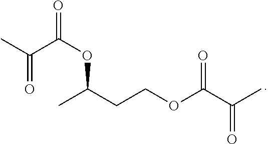 [SMILES: CC(=O)C(=O)OCC[C@@H](C)OC(=O)C(C)=O]

In a specific embodiment, diPyKe is (R)-butane-1,3-diyl bis(2-oxopropanoate).

Example 1-2 show synthesis of diLaKe compounds (formula II).

Example 3-5 describe administration of diLaKe (formula II) to rats following oral administration.

Examples 6-7 show chemical synthesis LaKe compounds XXII, XXIII, XXVIII, and XXIX.

Example 8 shows chemical synthesis of DiLa compounds XXVI and XXVII.

Example 9-10 show that administration of LaKe (formula XXII and XXIII), compared to a control, gives rise to an increased serum concentration of both BHB and lactate in rats following oral administration.

Example 11 shows that oral administration of LaKe (formula XXII and XXIII) leads to a decreased concentration of FFA in rat serum compared to the control.

Example 12 shows that administration with KeLa (formula XXIV and XXV), compared to a control, gives rise to an increased serum concentration of both BHB and lactate in rats following oral administration.

Example 13 shows that administration with DiLa (formula XXVI and XXVII), compared to a control, gives rise to an increased serum concentration of lactate in rats following oral administration.

Example 14 shows that oral administration of KeLa (formula XXIV and XXV), leads to a decreased concentration of FFA in rat serum compared to the control.

Example 15 shows that oral administration of DiLa (formula XXVI and XXVII), does not lead to a decreased concentration of FFA in rat serum compared to the control.

Example 16-17 show Chemoenzymatic synthesis of LaKe (formula XXII and XXIII) and DiLa (formula XXVI and XXVII) compounds.

Example 18 shows synthesis of R-(−)-β-hydroxybutyric acid esters (KeLa esters) (formula XXIV and XXV).

Example 19-20 shows a chemical or chemoenzymatic synthesis of diLaKe

Example 21 shows that oral administration with diLaKe, gives rise to an increased serum concentration of both BHB and lactate in rats.

Example 22 shows that oral administration of diLaKe leads to a decreased concentration of FFA in rat serum.

Example 23 shows that oral administration of diLaKe (formula II) gives rise to increased serum concentrations of Lac-Phe.

Example 24 shows a chemical synthesis of diPyKe (formula LXII).

Example 25 shows that oral administration of diPyKe gives rise to an increased serum concentration of both BHB and lactate in a rat.

Example 26 shows that oral administration of diPyKe (formula LXII) gives rise to increased serum concentrations of Lac-Phe.

Thus, one aspect of the invention relates to a compound of formula I

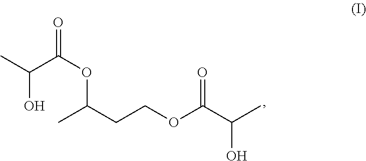 [SMILES: CC(CCOC(=O)C(C)O)OC(=O)C(C)O]

or a pharmaceutical acceptable salt thereof.

Another aspect of the invention relates to a compound of formula II or formula LXII

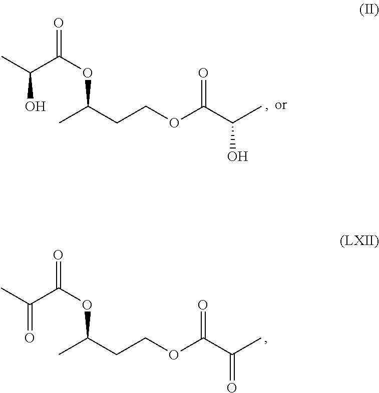

or a pharmaceutical acceptable salt thereof.

Another aspect of the present invention relates to a composition comprising a compound as described herein.

Yet another aspect of the present invention is to provide a pharmaceutical composition comprising the compound as described herein or a composition as described herein.

Still another aspect of the present invention is to provide a compound as described herein, a composition as described herein or a pharmaceutical composition as described herein for use as a medicament.

A further aspect of the present invention is to provide a food ingredient comprising the compound as described herein or a composition as described herein.

An even further aspect of the present invention is to provide a food product comprising the food ingredient as described herein.

A yet further aspect of the present invention is the use of the compound as described herein or the composition as described herein for non-therapeutic treatment.

### BRIEF DESCRIPTION OF THE FIGURES

FIG. 1

FIG. 1 shows the concentration (UM) of BHB (FIG. 1A) and lactate (FIG. 1B) in rat serum over time, following oral administration of either compound XXII and XXIII (LaKe) or physiological saline solution (0.9% NaCl) as a control to rats.

FIG. 2

FIG. 2 shows the concentration (UM) of non-esterified free fatty acids in rat serum over time following oral administration of either compound XXII and XXIII (LaKe) or physiological saline solution (0.9% NaCl) as a control to rats.

FIG. 3

FIG. 3 shows the concentration (UM) of BHB (FIG. 3A) and lactate (FIG. 3B) in rat serum over time, following oral administration of either compound XXIV and XXV (KeLa) or physiological saline solution (0.9% NaCl) as a control to rats. The increase in lactate for the KeLa ester is statistically significant from 90 min and onwards.

FIG. 4

FIG. 4 shows the concentration (UM) of BHB (FIG. 4A) and lactate (FIG. 4B) in rat serum over time, following oral administration of either compound XXVI and XXVII (DiLa) or physiological saline solution (0.9% NaCl) as a control to rats.

FIG. 5

FIG. 5 shows the concentration (UM) of Non-esterified free fatty acids in rat serum over time following oral administration of either compound XXIV and XXV (KeLa) or physiological saline solution (0.9% NaCl) as a control to rats.

FIG. 6

FIG. 6 shows the concentration (UM) of Non-esterified free fatty acids in rat serum over time following oral administration of either compound XXVI and XXVII (DiLa) or physiological saline solution (0.9% NaCl) as a control to rats.

FIG. 7

FIG. 7 shows the concentration (UM) of BHB (FIG. 7A) and lactate (FIG. 7B) in rat serum over time, following oral administration of compound II (diLaKe) to rats.

FIG. 8

FIG. 8 shows the concentration (UM) of non-esterified free fatty acids in rat serum over time, following oral administration of compound II (diLaKe) to rats.

FIG. 9

FIG. 9 shows the concentration (nM) of Lac-Phe in rat serum over time, following oral administration of compound II (diLaKe) to rats.

FIG. 10

FIG. 10 shows concentration (UM) of BHB (FIG. 10A) and lactate (FIG. 10B) in rat serum over time following oral administration of compound LXII (diPyKe) to a rat.

FIG. 11

FIG. 11 shows the concentration (nM) of Lac-Phe in rat serum over time, following oral administration of compound LXII (diPyKe) to a rat.

The present invention will now be described in more detail in the following.

### DETAILED DESCRIPTION OF THE INVENTION

### Definitions

Prior to discussing the present invention in further details, the following terms and conventions will first be defined:

### Ketone Body

In the present context the term “ketone body” refers to a water-soluble molecule containing a ketone group or the specific compounds BHB, acetoacetate and acetone. The compound is produced by the liver from fatty acids.

### Non-Esterified Free Fatty Acids (FFA)

In the present context, the term “Non-esterified free fatty acid” refers to metabolic fuel e.g. organic acids derived from hydrolysis of endogenous triglycerides. Increased levels of free fatty acids is associated with increased risk of disease.

### Oral Administration

In the present context, the term “Oral administration” refers to a route of administration, where the substance is administered through the mouth of the subject.

### Subject

The term “subject” comprises humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds. Preferred subjects are humans.

The term “subject” also includes healthy subjects of the population.

### Food

In the present context the term “food” or “food ingredient” is to be understood as also covering “feed” and “feed ingredient”. Thus, the food according to the invention may refer both to food/feed suitable for human and/or animal consumption and the term food ingredient according to the invention may refer both to food/feed suitable for human and/or animal consumption.

Generally speaking, the term “feed” refers to animal consumption and the term “food” refers to human or animal consumption. In the broad sense of the invention the terms may be used interchangeably.

### Nutraceutical

In the present context, the term “nutraceutical” or “bioceutical”, is to be understood as any substance that is a food or part of a food and may provide medical or health benefits, including the prevention and treatment of disease.

A “nutraceutical” or “bioceutical” is a pharmaceutical alternative, which claims physiological benefits. In the US, “nutraceuticals” are largely unregulated, as they exist in the same category as dietary supplements and food additives by the FDA, under the authority of the Federal Food, Drug, and Cosmetic Act.

In a preferred embodiment of the present invention, the compound or composition according to the invention, is a nutraceutical or is part of a nutraceutical.

In another preferred embodiment of the present invention, the compound or composition according to the invention, is a pharmaceutical or part of a pharmaceutical.

### Lactate/Ketone Body Esters

The present invention provides a novel group of Lactate/Ketone body esters and synthesis thereof for oral use in animals and humans. The compounds provide the beneficial properties known from lactate and BHB, while preventing the toxic ion load associated with the use of the individual compounds.

Furthermore, the compounds according to the present invention provide a unique solution for elevating serum concentrations of lactate, such as (S)-lactate, and BHB, such as (R)-BHB, in situations that require delivery of relatively more lactate than BHB.

Thus, a first aspect of the present invention relates to a compound of formula I

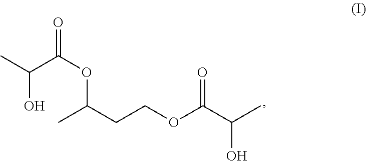 [SMILES: CC(CCOC(=O)C(C)O)OC(=O)C(C)O]

or a pharmaceutical acceptable salt thereof.

The compound of formula I comprises asymmetric C atoms and can therefore be presented in different isoforms.

In a preferred embodiment, the present invention relates to a compound of formula II

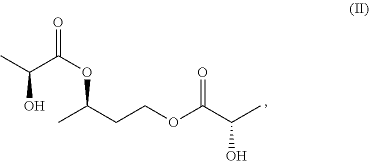 [SMILES: C[C@H](CCOC(=O)[C@H](C)O)OC(=O)[C@H](C)O]

or a pharmaceutical acceptable salt thereof.

In a further embodiment, the present invention relates to a compound of formula III

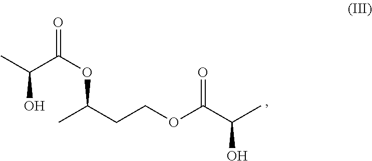 [SMILES: C[C@H](CCOC(=O)[C@@H](C)O)OC(=O)[C@H](C)O]

or a pharmaceutical acceptable salt thereof.

In a further embodiment, the present invention relates to a compound of formula IV

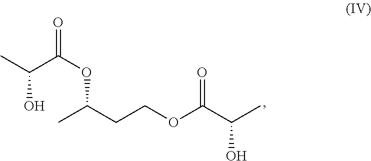 [SMILES: C[C@H](O)C(=O)OCC[C@H](C)OC(=O)[C@@H](C)O]

or a pharmaceutical acceptable salt thereof.

In a further embodiment, the present invention relates to a compound of formula V

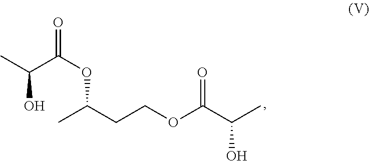 [SMILES: C[C@H](O)C(=O)OCC[C@H](C)OC(=O)[C@H](C)O]

or a pharmaceutical acceptable salt thereof.

In a further embodiment, the present invention relates to a compound of formula VI

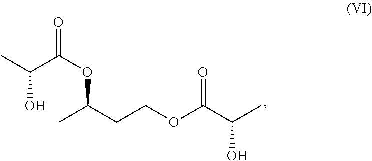 [SMILES: C[C@H](CCOC(=O)[C@H](C)O)OC(=O)[C@@H](C)O]

or a pharmaceutical acceptable salt thereof.

In a further embodiment, the present invention relates to a compound of formula VII

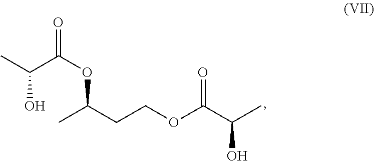 [SMILES: C[C@H](CCOC(=O)[C@@H](C)O)OC(=O)[C@@H](C)O]

or a pharmaceutical acceptable salt thereof.

In a further embodiment, the present invention relates to a compound of formula VIII

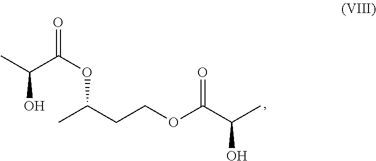 [SMILES: C[C@H](O)C(=O)O[C@@H](C)CCOC(=O)[C@@H](C)O]

or a pharmaceutical acceptable salt thereof.

In a further embodiment, the present invention relates to a compound of formula IX

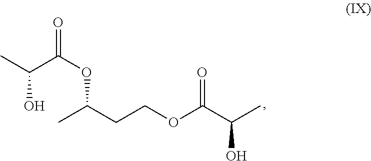 [SMILES: C[C@@H](CCOC(=O)[C@@H](C)O)OC(=O)[C@@H](C)O]

or a pharmaceutical acceptable salt thereof.

Lastly, the present invention also provides another related novel group of pyruvate/Ketone body esters and synthesis thereof for oral use in animals and humans. Without being bound by theory, the compounds provide the beneficial properties as above, since the pyruvate released in the body is converted into lactate. Additionally, the compound is an important intermediate in some processes for generating diLaKe.

Thus, another aspect of the present invention relates to a compound of formula LXI

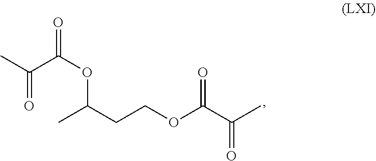 [SMILES: CC(=O)C(=O)OCCC(C)OC(=O)C(C)=O]

or a pharmaceutical acceptable salt thereof.

The compound of formula LXI comprises asymmetric C atoms and can therefore be presented in different isoforms.

In a preferred embodiment, the present invention relates to a compound of formula LXII

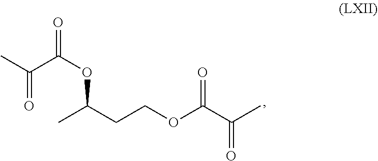 [SMILES: CC(=O)C(=O)OCC[C@@H](C)OC(=O)C(C)=O]

or a pharmaceutical acceptable salt thereof.

In another preferred embodiment, the present invention relates to a compound of formula LXIII

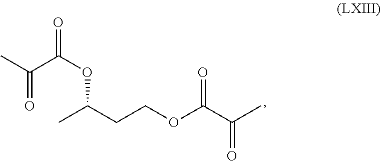 [SMILES: CC(=O)C(=O)OCC[C@H](C)OC(=O)C(C)=O]

or a pharmaceutical acceptable salt thereof.

### Composition

The compounds according to the present invention can be used in a composition together with other compounds such as other Lactate/Ketone body esters.

Thus, a further aspect of the present invention relates to a composition comprising the compound according to the present invention. In one embodiment, the present invention relates to a composition comprising a compound of formula II. In one embodiment, the present invention relates to a composition comprising a compound of formula LXII. In one embodiment, the present invention relates to a composition comprising a compound of formula II and a compound of formula LXII.

In a further embodiment, the composition further comprises one or more compounds selected from the group consisting of:
a compound with the formula X

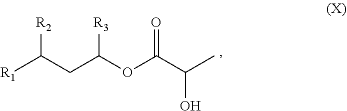

a compound of the formula XI

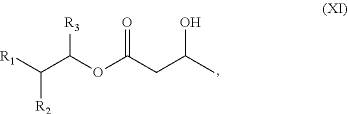

and
a compound of formula XII

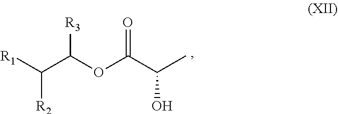

or a pharmaceutical acceptable salt thereof,
wherein R₁ is CH₃ or OH, R₂ is OH or H and R₃ is CH₃ or H.

In a further embodiment, the composition further comprises one or more compounds selected from the group consisting of a compound of:
a compound with the formula X

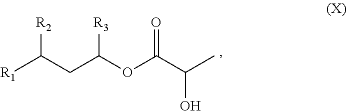

a compound of the formula XI

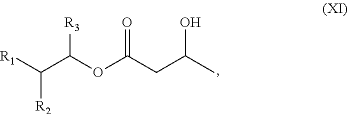

and
a compound of formula XII

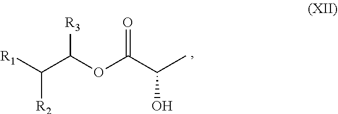

or a pharmaceutical acceptable salt thereof,
wherein R₁, R₂ and R₃ are CH₃, OH and H respectively or R₁, R₂ and R₃ are OH, H and CH₃ respectively.

Formula X, XI and XII have been tested in examples 9-15.

In a further embodiment, the composition further comprises one or more compounds selected from the group consisting of:
a compound with the formula XIII

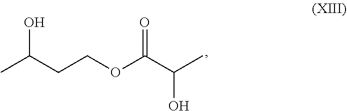

a compound with the formula XIV

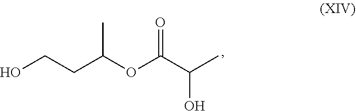

a compound with the formula XV

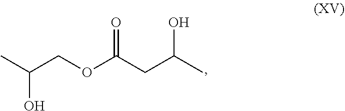

a compound with the formula XVI

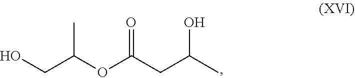

a compound with the formula XVII

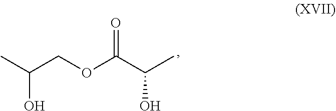

and
a compound with the formula XVIII

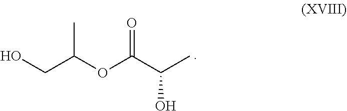

The compound of formula X, formula XI and formula XII can contain different asymmetric C atoms and can therefore be presented in different isoforms.

In a further embodiment, the composition further comprises one or more compounds selected from the group consisting of:
a compound with the formula XIX

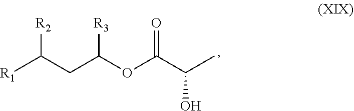

a compound of the formula XX

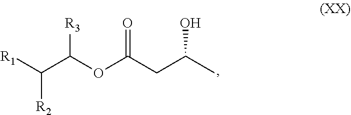

and
a compound of formula XXI

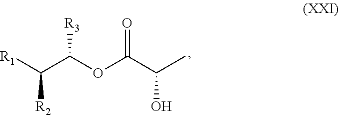

or a pharmaceutical acceptable salt thereof,
wherein R₁, R₂ and R₃ are CH₃, OH and H respectively or R₁, R₂ and R₃ are OH, H and CH₃ respectively.

In a further embodiment, the composition further comprises one or more compounds selected from the group consisting of:
a compound with formula XXII

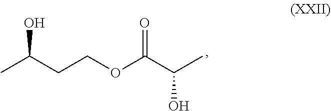

a compound with formula XXIII

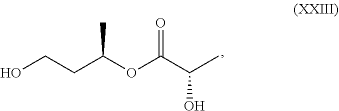

a compound with formula XXIV

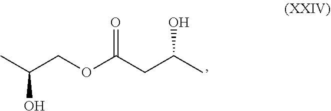

a compound with formula XXV

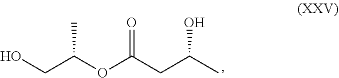

a compound with formula XXVI

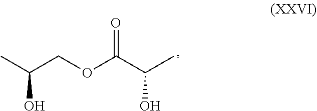

a compound with formula XXVII

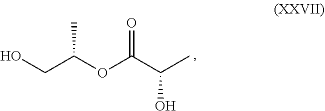

a compound with formula XXVIII

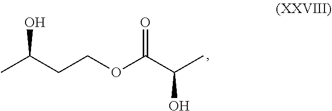

and
a compound with formula XXIX

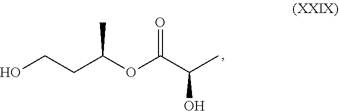

or pharmaceutical acceptable salts thereof.

In an even further embodiment, the composition further comprises a compound of the formula XXXII

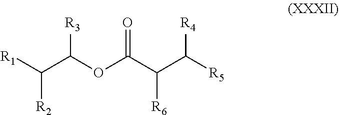

or a pharmaceutical acceptable salt thereof, wherein R₁ is H, CH₃, OH, CH₂OH or CH(CH₃)OH, R₂ is H or OH, R₃ is H or CH₃, R₄ and R₅ are H, OH or CH₃ and R₆ is H or OH.

In an embodiment, R₁ is CH(CH₃)OH, R₄ and R₅ are H, and R₆ is OH. In yet an embodiment, R₁ is CH(CH₃)OH, R₄ and R₅ are H, R₆ is OH, and R₂ is H. In an embodiment of the present invention, R₁ is CH(CH₃)OH. In another embodiment of the present invention, R₂ is H. In yet another embodiment of the present invention, R₃ is H. In an embodiment, R₁ is CH₂OH, R₄ and R₅ are H, and R₆ is OH. In yet an embodiment, R₁ is CH₂OH, R₄ and R₅ are H, R₆ is OH, and R₂ is H. In a further embodiment of the present invention, R₄ is H. In yet a further embodiment of the present invention, R₅ is H. In an even further embodiment of the present invention, R₆ is OH. In another embodiment, R₁ is CH₂OH. In a further embodiment, R₃ is CH₃. In another embodiment, R₁ is CH₃. In yet another embodiment, R₂ is OH. In a preferred embodiment, R₁ is CH₃, R₂ is OH, R₃ is H, R₄ and R₅ are H, and R₆ is OH. In another embodiment, R₁ is OH. In a preferred embodiment, R₁ is OH, R₂ is H, R₃ is CH₃, R₄ and R₅ are H, and R₆ is OH. In an embodiment of the present invention, R₄ is CH₃. In another embodiment of the present invention, R₄ is OH. In a further embodiment of the present invention, R₅ is CH₃. In yet a further embodiment of the present invention, R₅ is OH. In an even further embodiment of the present invention, R₆ is H.

In an even further embodiment, the composition further comprises a compound of the formula XXXIII

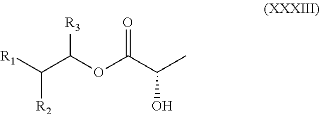

or a pharmaceutical acceptable salt thereof, wherein R₁ is H, CH₃, OH, CH₂OH or CH(CH₃)OH, R₂ is H or OH, R₃ is H or CH₃,

In an even further embodiment, the composition further comprises a compound of the formula XXXIV

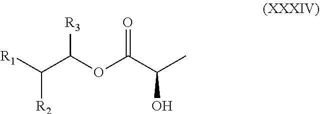

or a pharmaceutical acceptable salt thereof, wherein R₁ is H, CH₃, OH, CH₂OH or CH(CH₃)OH, R₂ is H or OH, R₃ is H or CH₃.

In an even further embodiment, the composition further comprises a compound of the formula XXXV

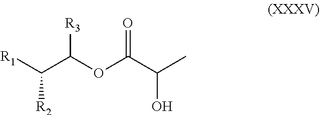

or a pharmaceutical acceptable salt thereof, wherein R₁ is H, CH₃, OH, CH₂OH or CH(CH₃)OH, R₂ is H or OH, R₃ is H or CH₃, wherein R₁ and R₂ are different and not H.

In an even further embodiment, the composition further comprises a compound of the formula XXXVI

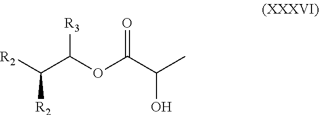

or a pharmaceutical acceptable salt thereof, wherein R₁ is H, CH₃, OH, CH₂OH or CH(CH₃)OH, R₂ is H or OH, R₃ is H or CH₃, wherein R₁ and R₂ are different and not H.

In an even further embodiment, the composition further comprises a compound of the formula XXXVII

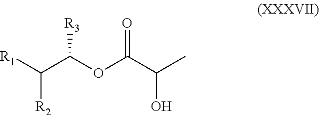

or a pharmaceutical acceptable salt thereof, wherein R₁ is H, CH₃, OH, CH₂OH or CH(CH₃)OH, R₂ is H or OH, R₃ is CH₃, wherein R₁ and R₂ are different and not H.

In an even further embodiment, the composition further comprises a compound of the formula XXXVIII

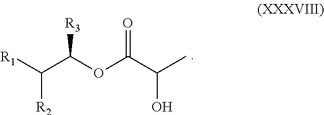

or a pharmaceutical acceptable salt thereof, wherein R₁ is H, CH₃, OH, CH₂OH or CH(CH₃)OH, R₂ is H or OH, R₃ is CH₃, wherein R₁ and R₂ are different and not H.

In an even further embodiment, the composition further comprises a compound of the formula XXXIX

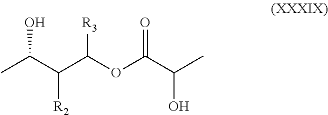

or a pharmaceutical acceptable salt thereof, wherein R₂ is H or OH, R₃ is H or CH₃.

In an even further embodiment, the composition further comprises a compound of the formula XXXX

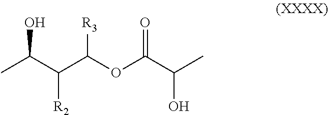

or a pharmaceutical acceptable salt thereof, wherein R₂ is H or OH, R₃ is H or CH₃.

In an even further embodiment, the composition further comprises a compound of the formula XXXXI

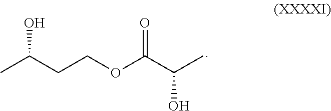

In an even further embodiment, the composition further comprises a compound of the formula XXXXII

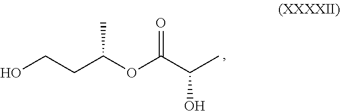

In an even further embodiment, the composition further comprises a compound of the formula XXXXIII

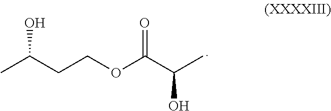

In an even further embodiment, the composition further comprises a compound of the formula XXXXIV

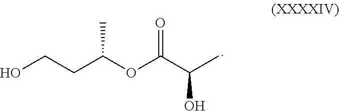

In an even further embodiment, the composition further comprises a compound of the formula XXXXV

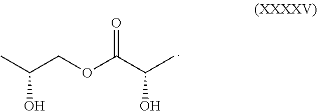

In an even further embodiment, the composition further comprises a compound of the formula XXXXVI

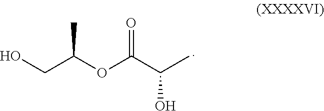

In an even further embodiment, the composition further comprises a compound of the formula XXXXVII

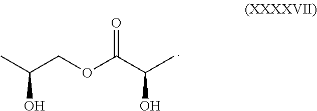

In an even further embodiment, the composition further comprises a compound of the formula XXXXVIII

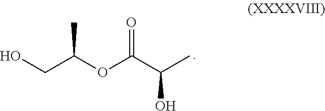

In an even further embodiment, the composition further comprises a compound of the formula XXXXIX

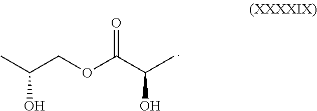

In an even further embodiment, the composition further comprises a compound of the formula L

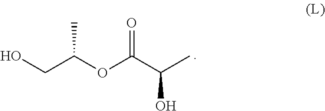

The compounds according to the present invention and the further compounds as described above can be used in a composition comprising different amounts of the described compounds. The compound according to the present invention leads to two lactate molecules per BHB, thus, combining this compound with other compounds as described herein results in combinations having different release profiles of lactate in relation to BHB.

In a further embodiment, the composition comprises a compound of formula I and a compound of formula XXII.

In a further embodiment, the composition comprises a compound of formula I and a compound of formula XXIII.

In a further embodiment, the composition comprises a compound of formula I, a compound of formula XXII and a compound of formula XXIII.

In a still further embodiment, the present invention relates to a composition comprising a compound according to formula I and a compound according to formula XXII and/or XXIII, such as in a ratio of formula I to formula XXII and/or XXIII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula II and a compound of formula XXII.

In yet a further embodiment, the composition comprises a compound of formula II and a compound of formula XXIII.

In yet a further embodiment, the composition comprises a compound of formula II, a compound of formula XXII and a compound of formula XXIII.

In one embodiment, the present invention relates to a composition comprising a compound according to formula II and a compound according to formula XXII and/or XXIII, such as in a ratio of formula II to formula XXII and/or XXIII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In a further embodiment, the composition comprises a compound of formula I and a compound of formula XXIV.

In a further embodiment, the composition comprises a compound of formula I and a compound of formula XXV.

In a further embodiment, the composition comprises a compound of formula I, a compound of formula XXIV and a compound of formula XXV.

In a still further embodiment, the present invention relates to a composition comprising a compound according to formula I and a compound according to formula XXIV and/or XXV, such as in a ratio of formula I to formula XXIV and/or XXV in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula II and a compound of formula XXIV.

In yet a further embodiment, the composition comprises a compound of formula II and a compound of formula XXV.

In yet a further embodiment, the composition comprises a compound of formula II, a compound of formula XXIV and a compound of formula XXV.

In one embodiment, the present invention relates to a composition comprising a compound according to formula II and a compound according to formula XXIV and/or XXV, such as in a ratio of formula II to formula XXIV and/or XXV in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In a further embodiment, the composition comprises a compound of formula I and a compound of formula XXVI.

In a further embodiment, the composition comprises a compound of formula I and a compound of formula XXVII.

In a further embodiment, the composition comprises a compound of formula I, a compound of formula XXVI and a compound of formula XXVII.

In a still further embodiment, the present invention relates to a composition comprising a compound according to formula I and a compound according to formula XXVI and/or XXVII, such as in a ratio of formula I to formula XXVI and/or XXVII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula II and a compound of formula XXVI.

In yet a further embodiment, the composition comprises a compound of formula II and a compound of formula XXVII.

In yet a further embodiment, the composition comprises a compound of formula II, a compound of formula XXVI and a compound of formula XXVII.

In one embodiment, the present invention relates to a composition comprising a compound according to formula II and a compound according to formula XXVI and/or XXVII, such as in a ratio of formula II to formula XXVI and/or XXVII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In a further embodiment, the composition comprises a compound of formula LXI and a compound of formula XXII.

In a further embodiment, the composition comprises a compound of formula LXI and a compound of formula XXIII.

In a further embodiment, the composition comprises a compound of formula LXI, a compound of formula XXII and a compound of formula XXIII.

In a still further embodiment, the present invention relates to a composition comprising a compound according to formula LXI and a compound according to formula XXII and/or XXIII, such as in a ratio of formula LXI to formula XXII and/or XXIII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula LXII and a compound of formula XXII.

In yet a further embodiment, the composition comprises a compound of formula LXII and a compound of formula XXIII.

In yet a further embodiment, the composition comprises a compound of formula LXII, a compound of formula XXII and a compound of formula XXIII.

In one embodiment, the present invention relates to a composition comprising a compound according to formula LXII and a compound according to formula XXII and/or XXIII, such as in a ratio of formula LXII to formula XXII and/or XXIII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In a further embodiment, the composition comprises a compound of formula LXI and a compound of formula XXIV.

In a further embodiment, the composition comprises a compound of formula LXI and a compound of formula XXV.

In a further embodiment, the composition comprises a compound of formula LXI, a compound of formula XXIV and a compound of formula XXV.

In a still further embodiment, the present invention relates to a composition comprising a compound according to formula LXI and a compound according to formula XXIV and/or XXV, such as in a ratio of formula LXI to formula XXIV and/or XXV in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula LXII and a compound of formula XXIV.

In yet a further embodiment, the composition comprises a compound of formula LXII and a compound of formula XXV.

In yet a further embodiment, the composition comprises a compound of formula LXII, a compound of formula XXIV and a compound of formula XXV.

In one embodiment, the present invention relates to a composition comprising a compound according to formula LXII and a compound according to formula XXIV and/or XXV, such as in a ratio of formula LXII to formula XXIV and/or XXV in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In a further embodiment, the composition comprises a compound of formula LXI and a compound of formula XXVI.

In a further embodiment, the composition comprises a compound of formula LXI and a compound of formula XXVII.

In a further embodiment, the composition comprises a compound of formula LXI, a compound of formula XXVI and a compound of formula XXVII.

In a still further embodiment, the present invention relates to a composition comprising a compound according to formula LXI and a compound according to formula XXVI and/or XXVII, such as in a ratio of formula LXI to formula XXVI and/or XXVII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In yet a further embodiment, the composition comprises a compound of formula LXII and a compound of formula XXVI.

In yet a further embodiment, the composition comprises a compound of formula LXII and a compound of formula XXVII.

In yet a further embodiment, the composition comprises a compound of formula LXII, a compound of formula XXVI and a compound of formula XXVII.

In one embodiment, the present invention relates to a composition comprising a compound according to formula LXII and a compound according to formula XXVI and/or XXVII, such as in a ratio of formula LXII to formula XXVI and/or XXVII in the range 100:1 to 1:100, such as in the ratio 50:1 to 1:50, such as in the ratio 25:1 to 1:25, such as in the ratio 5:1 to 1:5 or in the range 100:1 to 1:1, such as in the ratio 50:1 to 1:1, preferably in the ratio 25:1 to 1:1, more preferably in the ratio 25:1 to 5:1.

In addition to the compound disclosed in relation to the present invention, the composition may further comprise other ketone esters or ketonebody precursors.

Thus, in one embodiment of the present invention, the composition further comprises one or more ketone esters or ketonebody precursors different from compounds according to any of formula I to XXIX and XXXII to L, such as any of formula I to XXIX. In another embodiment of the present invention, the composition further comprises one or more ketone esters or ketonebody precursors different from compounds according to any of formula LXI to LXIII.

In another embodiment of the present invention, the one or more ketone ester or ketonebody precursors different from compounds according to any of formula I to XXIX and XXXII to L, such as any of formula I to XXIX, is selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate. In another embodiment of the present invention, the one or more ketone ester or ketonebody precursors different from compounds according to any of formula LXI to LXIII, is selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.

In another embodiment of the present invention, the composition as described herein comprises a compound of formula II and at least one compound selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.

In another embodiment of the present invention, the composition as described herein comprises a compound of formula LXII and at least one compound selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.

In yet a further embodiment, the composition comprises a compound of formula II and (3R)-hydroxybutyl (3R)-hydroxybutyrate. In yet a further embodiment, the composition comprises a compound of formula II and 1,3-butanediol dihexanoate. In yet a further embodiment, the composition comprises a compound of formula II and 1,3-butanediol. In yet a further embodiment, the composition comprises a compound of formula LXII and (3R)-hydroxybutyl (3R)-hydroxybutyrate. In yet a further embodiment, the composition comprises a compound of formula LXII and 1,3-butanediol dihexanoate. In yet a further embodiment, the composition comprises a compound of formula LXII and 1,3-butanediol.

In a further embodiment, the composition according to the invention, further comprises a dietetically and/or pharmaceutically acceptable carrier.

In yet another embodiment, the composition according to the invention, further comprising a sugar carbohydrate.

### Uses

BHB and lactate have been shown to ameliorate or be useful as treatment for a number of different diseases. This both due to their antiinflammatory effects etc., as well as their general effect as high-energy substrates. Furthermore, they will be relevant in relation to endurance or sports performance e.g. via improvement of muscle output/motor function.

Thus an aspect of the present invention relates to a pharmaceutical composition comprising the compounds according to the present invention or the composition according to the present invention.

Thus, in one embodiment the compound as described herein, the composition as described herein or the pharmaceutical composition as described herein for use as a medicament.

BHB and lactate have been associated with beneficial outcome when used in the treatment of inflammatory disease, cancer, epileptic seizures, acute heart failure, Resuscitation, acidosis, traumatic brain injury, acute pancreatitis, hepatitis, myocardial infarction, burns, sepsis, dengue, cognition, sarcopenia, atherosclerosis, neurodegeneration, oxidative stress and wound healing. Even further BHB and lactate have been associated with beneficial outcome when used in the treatment of chronic heart failure, cardiogenic shock, myocardial ischemia, pulmonary hypertension, systemic hypertension, organ transplantation preparation, post-organ transplantation reperfusion injury, and multiple sclerosis.

The compounds of formula XXII, XXIII, XXIV, XXV, XXVI and XXVII are able to increase circulating levels of ketone body BHB and/or lactate (see examples 10, 12 and 13). The compounds of formula II, and LXII are able to increase circulating levels of ketone body BHB and/or lactate (see examples 21, and 25).

Thus, in one embodiment of the present invention the compounds as described herein, the compositions as described herein or the pharmaceutical compositions as described herein can be used in the treatment, prevention or alleviation of inflammatory disease, cancer, epileptic seizures, acute heart failure, Resuscitation, acidosis, traumatic brain injury, acute pancreatitis, hepatitis, myocardial infarction, burns, sepsis, dengue, cognitive dysfunction, atherosclerosis, neurodegeneration, oxidative stress, Alzheimer's disease, Parkinson's disease, amyotrophic lateral sclerosis, epilepsy, astrocytoma, glioblastoma and Huntington's chorea, sarcopenia, muscle atrophy and dysfunctional wound healing. In another embodiment of the present invention the compounds as described herein, the compositions as described herein or the pharmaceutical compositions as described herein can be used in the treatment, prevention or alleviation of inflammatory disease, cancer, epileptic seizures, acute and chronic heart failure, cardiogenic shock, resuscitation, acidosis, traumatic brain injury, acute pancreatitis, hepatitis, myocardial infarction, atherosclerosis, ischemic diseases, myocardial ischemia, pulmonary hypertension, systemic hypertension, organ transplantation preparation, post-organ transplantation reperfusion injury, burns, sepsis, dengue, cognitive dysfunction, atherosclerosis, neurodegeneration, oxidative stress, Alzheimer's disease, Parkinson's disease, multiple sclerosis, amyotrophic lateral sclerosis, epilepsy, astrocytoma, glioblastoma and Huntington's chorea, sarcopenia, muscle atrophy, osteoporosis, dysfunctional wound healing, tissue after injury, such as facilitating tissue repair following injury, and infertility.

In yet an embodiment of the present invention, the compounds as described herein, the compositions as described herein or the pharmaceutical compositions as described herein can be used in the treatment, prevention or alleviation of elevated plasma levels of free fatty acids in a human or animal subject; cognitive dysfunction, neurodegenerative diseases, for instance Alzheimer's disease, Parkinson's disease, Huntington's chorea, epilepsy; hypoxic states, for instance angina pectoris, extreme physical exertion, intermittent claudication, hypoxia, stroke and myocardial infarction; insulin resistant states, for instance infection, stress, obesity, diabetes, metabolic syndrome and heart failure; inflammatory states including infection and autoimmune disease and muscle impairment, fatigue and muscle fatigue. It is further disclosed that the compound reduces plasma levels of fatty acids and may be used for e.g. treating a condition, which is caused by, exacerbated by or associated with elevated plasma levels of free fatty acids in a human or animal subject.

The compounds of formula XXII, XXIII, XXIV and XXV are able to lower the level of free fatty acids (FFA) in blood (see examples 11 and 14). The compound of formula II is able to lower the level of free fatty acids (FFA) in blood (see example 22). Without wishing to be bound by theory, the compound of formula LXII, will have the same effect (example 25).

The level of FFA in blood reflects ongoing lipolysis and high levels of FFA induce insulin resistance; conversely agents that lower the level of free fatty acids in the blood can be used therapeutically to increase insulin sensitivity in people with type 2 diabetes and the metabolic syndrome (6).

Thus, in one embodiment of the present invention, the compounds, composition or pharmaceutical composition according to the invention can be used in the treatment, prevention or alleviation of a condition, which is caused by, exacerbated by or associated with, elevated plasma levels of free fatty acids in a human or animal subject.

N-lactoyl-phenylalanine (Lac-Phe) is a lactate-derived metabolite formed by the enzyme CDNP2. Chronic administration of Lac-Phe to diet-induced obese mice decreases adiposity and body weight and improves glucose homeostasis. The compound of formula II is able to increase the level of Lac-Phe in blood (see example 23). Without wishing to be bound by theory, the compound of formula LXII, will have the same effect (example 26).

Thus in one embodiment of the present disclosure the compounds, composition or pharmaceutical composition according to the invention can be used to increase levels of appetite-supressing hormones, such as N-lactoyl-phenylalanine (Lac-Phe). In one embodiment of the present invention, the compounds, composition or pharmaceutical composition according to the invention can be used in the treatment, prevention or alleviation of a condition, by increasing plasma levels of Lac-Phe in a human or animal subject. The method comprises administering to the subject a compound or composition according to the invention.

Thus, in a preferred embodiment of the present invention, the compounds can be used to control the level of free fatty acids in the blood of the subject.

In another embodiment of the present invention, the compounds as described herein, the compositions as described herein or the pharmaceutical compositions as described herein can be use in the treatment, prevention or alleviation of viral infections, immunogenic disorders, non-alcoholic fatty liver disease (NAFLD), non-alcoholic steatohepatitis (NASH), sarcopenia, muscle fatigue, angina pectoris, extreme physical exertion, intermittent claudication, hypoxia, stroke and myocardial infarction, stress, obesity, diabetes, metabolic syndrome, autoimmune disease, muscle impairment or to improve motor function. In another embodiment of the present invention, the compounds as described herein, the compositions as described herein or the pharmaceutical compositions as described herein can be use in the treatment, prevention or alleviation of viral infections, immunogenic disorders, non-alcoholic fatty liver disease (NAFLD), non-alcoholic steatohepatitis (NASH), sarcopenia, loss of muscle/lean body mass, muscle fatigue, angina pectoris, extreme physical exertion, intermittent claudication, hypoxia, stroke, CNS bleedings and myocardial infarction, stress, obesity, diabetes, metabolic syndrome, autoimmune disease, muscle impairment or to improve motor function.

In one embodiment, the compounds, composition or pharmaceutical composition according to the invention can be used use in the treatment, prevention and/or alleviation of insulin resistance.

In another embodiment, the compounds as described herein, the compositions as described herein or the pharmaceutical compositions as described herein can be used in the treatment of insulin resistance, preferably the compound can be used in increasing the insulin sensitivity in the subject.

In yet another embodiment, the compounds as described herein, the compositions as described herein or the pharmaceutical compositions as described herein can be used in a treatment to inhibit or alleviate inflammation, atherosclerosis, neurodegeneration, oxidative stress, carcinogenesis angiogenesis, obesity, diabetes, and metabolic syndrome. In yet another embodiment, the compounds as described herein, the compositions as described herein or the pharmaceutical compositions as described herein can be used in a treatment to inhibit or alleviate inflammation, atherosclerosis, neurodegeneration, oxidative stress, carcinogenesis angiogenesis, obesity, diabetes, and metabolic syndrome.

Administration of the compounds as described herein, the compositions as described herein or the pharmaceutical compositions as described herein can be done in a number of ways as described in the following, non-limiting examples. Oral or Nasal, which is administration through the mouth or nose. By intradermal injection, which is a delivery of the compound into the dermis of the skin, located between epidermis and the hypodermis. Alternatively, the compound can be administered intravenously, which is an administration directly into the blood stream of the subject. Further, intramuscular administration of the compound is an injection into the muscles of the subject. In addition, the compound can be administered subcutaneous, which is under the skin, in the area between the muscle and the skin of the subject. Further, the compound can be administered intratracheal, which is administration directly into the trachea and by transdermal administration, which is administration across the skin.

Any mode of administration can be used as long as the mode results in the desired effect of the compound.

Thus, in one embodiment, the compounds as described herein, the compositions as described herein or the pharmaceutical compositions as described herein is administered to the subject by oral or nasal administration.

In a preferred embodiment, the compounds as described herein, the compositions as described herein or the pharmaceutical compositions as described herein is administered to a subject by oral administration.

In another preferred embodiment, the compounds, composition or pharmaceutical composition according to the invention is orally administered.

The compounds as described herein, the compositions as described herein or the pharmaceutical compositions as described herein may be administered in doses suitable for providing the desired effect in the subject receiving the compound.

In one embodiment the compounds as described herein, the compositions as described herein or the pharmaceutical compositions as described herein is administered in a dose of 0.05-1.5 g/kg, preferably 0.15-1.5 g/kg, more preferably 0.2-1.0 g/kg, even more preferably 0.2-0.5 g/kg.

In another embodiment the compound, composition or pharmaceutical composition according to the invention is administered in a dose of 0.05-15 g/kg, preferably 0.15-10 g/kg, more preferably 0.2-5 g/kg, even more preferably 0.2-2.5 g/kg, even more preferably 0.2-1.5 g/kg.

In another embodiment the compound, composition or pharmaceutical composition according to the invention is administered in a daily dosage in the range 0.15-45 g/kg, preferably 0.45-30 g/kg, more preferably 0.6-15 g/kg or 0.6-4.5 g/Kg, even more preferably 0.6-1.5 g/kg.

The “subject” as described herein is supposed to receive the compound and comprises humans of all ages, other primates (e.g., cynomolgus monkeys, rhesus monkeys); mammals in general, including commercially relevant mammals, such as cattle, pigs, horses, sheep, goats, mink, ferrets, hamsters, cats, dogs; and/or birds in need of the described compounds. Preferred subjects are humans.

The term “subject” also includes healthy subjects of the population.

Thus, in an embodiment of the present invention, the subject is selected from the group consisting of; humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds.

In a preferred embodiment, the subject is a human.

In one embodiment, the subject being an elderly subject, such as a human above 40 years of age, such as above, 50, such as above 60 such as above 70 or such as above 80. In one embodiment, the subject has a high body mass index (BMI), such as a BMI>30. Thus, without being bound by theory, in some embodiments, a high BMI can be used to distinguish between therapeutic and non-therapeutic treatments.

In one embodiment, the compound of the present invention is administered as a part of a pharmaceutical composition.

The compound and the composition of the present invention can be in different forms, dry powder, aqueous solution, gel.

In one embodiment of the present invention the compounds as described herein, the compositions as described herein or the pharmaceutical compositions as described herein is an aqueous solution, gel or powder, preferably in an aqueous solution.

### Food Ingredients and Food Products

The compounds or the composition according to the present invention can be comprised in a food ingredient.

Thus, an aspect of the present invention relates to a food ingredient comprising the compound or the composition according to the invention.

Further, the food ingredient according to the invention may be part of a food product. Thus, an aspect of the present invention relates to a food product comprising a food ingredient according to the present invention.

In one embodiment of the present invention, the food is selected from the group consisting of a nutraceutical, a food supplement, a dietary supplement, a feed, bar, sugar bar, protein bar, powder, gel, beverage, drink, yoghurt, chewing gum, dairy product, sports drink, confectionary product, ice cream, capsule, tablet, sachet, and pouch.

### Non-Therapeutic Uses

The compounds or the compositions according to the invention may be used in a non-therapeutic treatment.

Thus, one aspect of the present invention relates to the use of the compound or composition according to the invention, to reduce free fatty acids circulating in the blood plasma of a subject, in the non-therapeutic treatment of muscle impairment, fatigue or improve motor function.

In one embodiment of the present invention, the compound or composition is for the non-therapeutic treatment of cardiac muscle fatigue, skeletal muscle fatigue, and/or improvement of motor function or for promoting alertness or improving cognitive function in a subject.

In one embodiment of the present invention, the compound or composition is for the non-therapeutic treatment of cardiac muscle fatigue, skeletal muscle fatigue, and/or improvement of motor function or for promoting alertness or improving cognitive function in a subject, or to prevent neurodegeneration in a subject.

In one embodiment of the present invention, the compound or composition can promote mental well being in a subject.

In one embodiment, the compositions for use to reduce free fatty acids circulating in the blood plasma of a subject, in the non-therapeutic treatment of muscle impairment or fatigue, do not comprise DiLa.

Thus, one aspect of the present invention relates to the use of the compound or composition according to the invention, to suppressing appetite, treating obesity, promoting weight loss, preventing, alleviating and/or treating sarcopenia, maintaining a healthy weight or decreasing the ratio of fat to lean muscle.

An aspect also relates to the use of a compound or composition according to the invention to non-therapeutically increasing serum concentration of Lac-Phe.

In one embodiment of the present invention, the compound or composition can increase longevity in a subject.

In one embodiment of the present invention, the compound or composition is for promoting wound healing and tissue repair following injury in a subject.

In one embodiment of the present invention, the compound or composition is for promoting bone health in a subject, such as in the non-therapeutic treatment of osteoporosis, such as non-therapeutically increasing the bone mass.

### Process

The compound can be produced either through chemoenzymatic synthesis as described in example 1 or example 20 or through chemical synthesis as seen in example 2, or example 19.

Thus, in one aspect the invention relates to a process for producing a compound according to the invention, the process comprising
a) providing a compound of formula XXX;

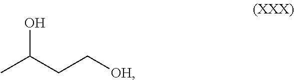

b) reacting said compound from step a) with ethyl lactate, such as (S)-(−)-ethyl lactate in the presence of a Lipase; and
c) providing a compound as described herein.

In one embodiment, the process according to the invention for producing the compound of formula II, the process comprising
a) providing a compound of formula XXXI;

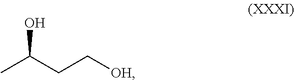

b) reacting said compound from step a) with ethyl lactate, such as (S)-(−)-ethyl lactate in the presence of a Lipase; and
c) providing a compound of formula II.

In one embodiment, the process further comprises a step d) of purifying the provided compounds, such as by filtering, distillation and/or flash column chromatography (FCC).

In another embodiment, the Lipase is immobilized on a solid support such as on beads.

Lipases catalyze the hydrolysis of triacylglycerols into glycerol and free fatty acids. Lipases may however also be used in other functions, such as in the process above.

Candida antarctica lipase B (CALB) possesses wide substrate specificity, high activity and high enantioselectivity, hence it is considered as a major enzyme in biotechnology. It also has the capability to perform in aqueous and non-aqueous reaction environments. CALB may be used in transesterification, kinetic resolution and polymerization reactions.

Thus, in another embodiment, the Lipase is selected from the group consisting of Lipase B, Lipase B Candida Antarctica immobilized on Immobead 150, Novozym® 435, CALB, CALB lipase immobilised on a hydrophobic carrier, Lipozyme TL IM and Lipozyme® RM, 1,3 specific lipase.

In a preferred embodiment, the Lipase is Lipase B, more preferably CALB or Novozym 435.

In the example section (example 1), Novozym 435 has been used. In the example section (examples 16 and 17), Lipase B Candida Antarctica immobilized on Immobead 150 has been used.

As provided by example 20, the inventors have devised an alternative chemoenzymatic process, which firstly provides diPyKe through a chemical synthesis step.

Thus, in another aspect the invention relates to a process for producing a compound according to the invention, the process comprising
a) providing 1,3 butanediol, such as (R)-1,3-butanediol, and pyruvic acid;
b) reacting said compounds from step a) in the presence of a catalytic acid, such as p-toluenesulfonic acid (pTsOH); and
c) obtaining, diPyKe, as described herein.

In one embodiment, step b is performed in a suitable solvent, such as in anhydrous toluene.

To further provide diLaKe, the process may comprise the additional hydrogenation step of reducing the ketones of diPyKe to alcohols. In one embodiment, the process comprises the step d) of reacting diPyKe from step c in the presence of a dehydrogenase enzyme. In one embodiment, the process comprises the step d) of reacting diPyKe from step c in the presence of a stoichiometric reducing agent or another method for hydrogenation, e.g. by catalytic hydrogenation.

Thus, such an aspect may be defined as a process for producing a compound according to the invention, the process comprising:
a) providing 1,3 butanediol, such as (R)-1,3-butanediol, and pyruvic acid;
b) reacting said compounds from step a) in the presence of a catalytic acid, such as p-toluenesulfonic acid (pTsOH);
c) obtaining diPyKe, as described herein;
d) reacting the compound of step c) in the presence of a reducing agent, such as a dehydrogenase, such as in the presence of bakers yeast;
e) obtaining diLaKe as described herein.

In one embodiment, the dehydrogenase enzyme is selected from the group consisting of an alcohol dehydrogenase, a lactate dehydrogenase, a pyruvate dehydrogenase, and a glutamate dehydrogenase. The dehydrogenase may be from yeast. Preferably, the dehydrogenase enzyme is purified such that it essentially consists of the purified enzyme. When a purified dehydrogenase enzyme, is used, it may further be necessary to include a suitable co-enzyme, such as NADH or NADPH for the reaction to function, and possibly means for rengerating the co-enzymes.

A source of dehydrogenases may also be provided by their presence in yeast cells, such as bakers yeast. Thus, in one embodiment, the dehydrogenase enzyme is bakers yeast, such as YSC2 from Sigma-Aldrich.

In one embodiment, the process further comprises a step of purifying the provided compounds, such as by filtering, distillation and/or flash column chromatography (FCC).

In a further aspect, the invention relates to a process for producing the compound, wherein

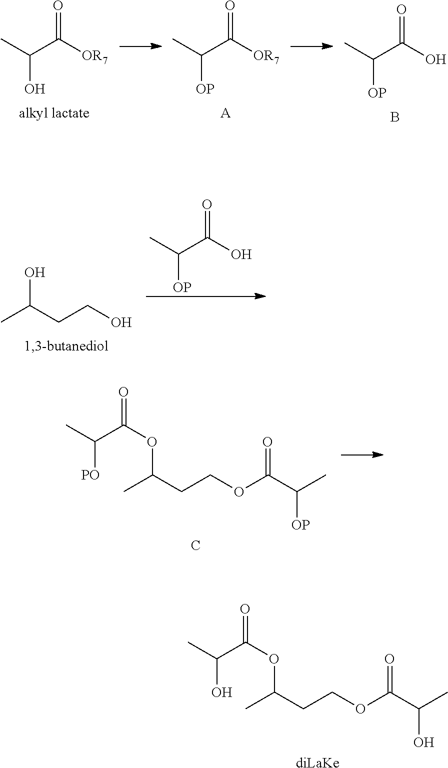

At first, an alkyl (R₇) lactate, as e.g. methyl, ethyl or propyl lactate, is converted into the compound of formula A. The alcohol group is protected with a protecting group (P) as known to persons skilled in the art e.g. by using TBSCl, Imidazole and DMF. P may be any protecting group as commonly known in the art. The skilled person will be aware of suitable protecting groups for hydroxyl groups, examples of which may be based on trialkylsilyl derivatives as e.g. trimethylsilyl (TMS), triethylsilyl (TES), tert-butyldimethylsilyl (TBS), tert-butyldiphenylsilyl (TBDPS) and triisopropylsilyl (TIPS). The protecting group may be based on groups that are removable under hydrogenative conditions, such as benzyl (Bn), para-methoxy-benzyl (p-OMe-Bn). The protecting group may be based on groups that are removable under acidic conditions, such a methoxymethyl (MOM), benzyloxymethyl (BOM).

In the second step, the compound of formula A is converted to a compound of formula B by hydrolysing the ester-group using an alkaline solution such as LiOH, NaOH, KOH, CsOH, K₃PO₄, Cs₂CO₃, dissolved in a suitable solvent. A suitable solvent for this reaction step may be tetrahydrofuran (THF), or ethanol. However, other solvents such as 2-methyl THF (2-Me-THF), water, alcohols such as ethanol or isopropanol, acetonitrile, 1,4-dioxane, dimethylsulfoxide (DMSO), N,N-dimethylformamide (DMF), diglyme (bis (2-methoxyethyl) ether), as well as mixtures of solvents, may also be used. After hydrolysis, the mixture is neutralised with an acid and the product B isolated using standard extraction procedures known to those skilled in the art.

Following, a compound of formula C is prepared by reacting 1,3-butanediol with the compound of formula B in the presence of coupling or dehydrating reagents, such as O-(Benzotriazol-1-yl)-N,N,N′,N′-tetramethyluronium tetrafluoroborate (TBTU), to form a compound of formula C. Any coupling reagent or dehydrating agent commonly known in the art can be used, such as, N,N′-dicyclohexylcarbodiimide (DCC), N,N′-diisopropylcarbodiimide (DIC), N-ethyl-N′-(3-dimethylaminopropyl) carbodimide (EDC), N,N′-Disuccinimidyl carbonate (DSC), 1-Cyano-2-ethoxy-2-oxoethylidenaminooxy)dimethylamino-morpholino-carbenium hexafluorophosphate (COMU), O-Benzotriazole-N,N,N′,N′-tetramethyluronium hexafluorophosphate (HBTU), Tetramethylfluoroformamidinium hexafluorophosphate (TFFH). This may also involve formation of an activated species, such as a mixed anhydride, from formula B by reacting it with a chloroformate or an acyl halide. The reaction may be performed in a solvent such as dimethylformamide (DMF) or other solvents as commonly known to the persons skilled in the art, and potentially further comprising an organic compound such as N,N-Diisopropylethylamine (DIPEA), triethylamine (TEA), 4-Dimethylaminopyridine (DMAP), or other bases as commonly known to the persons skilled in the art.

Lastly, the protecting groups (P) are removed from the compound of formula C by methods known in the art for the various protecting groups as defined herein, as an example TBS may be removed under acidic conditions, e.g. by KHSO₄, HCl, or by reaction with a fluoride source, such as tetrabutylammonium fluoride (TBAF). hydrogen fluoride, hydrogen fluoride-pyridine complex, triethylamine trihydrofluoride. A suitable solvent for this reaction step may be tetrahydrofuran (THF), methanol and water or mixtures thereof. However, other solvents, such as acetonitrile, 2-Me-THF, toluene, ethyl acetate, dichloromethane, either alone or as mixtures, may also be used. Another example is the removal of benzyl by adding palladium on carbon (Pd/C) together with a hydrogen source, such as H₂. A suitable solvent for this reaction step may be ethanol and water or mixtures thereof. However, other solvents, such as methanol, tetrahydrofuran (THF), 2-Me-THF, ethyl acetate (EtOAc), toluene, DMF, acetonitrile either alone or as mixtures, may also be used. Alternative metal catalysts, including their immobilized forms, such as platinum, rhodium or nickel may be used. Furthermore, an alternative method involves the use of transfer hydrogenation using 2-propanol and formate salts as the source of hydrogen.

Additionally, the final product (diLake) may be purified through methods that are commonly known in the art, e.g. by removing MeOH or EtOH under reduced pressure and adding water before extraction with EtOAc, collecting the organic layers and drying over Na₂SO₄, filtering and concentrating, followed by a flash column chromatography on silica gel, crystallization, precipitation or distillation.

### Other Aspects

US2011237666 A1 for example discloses numerous physical states or diseases, which may be treated with ketone compounds.

An aspect of the invention relates to a method of treating a condition, which is caused by, exacerbated by or associated with elevated plasma levels of free fatty acids in a human or animal subject, which method comprises administering to the subject a compound, composition or pharmaceutical composition according to the invention.

Another aspect of the invention relates to a method of treating a condition where weight loss or weight gain is implicated, which method comprises administering to a subject in need thereof a compound, composition or pharmaceutical composition according to the invention.

A further aspect of the invention relates to a method of suppressing appetite, treating obesity, promoting weight loss, maintaining a healthy weight or decreasing the ratio of fat to lean muscle, which method comprises administering to a subject in need thereof a compound, composition or pharmaceutical composition according to the invention.

Yet an aspect of the invention relates to a method of preventing or treating a condition selected from cognitive dysfunction, a neurodegenerative disease or disorder, muscle impairment, fatigue and muscle fatigue, which method comprises administering to a subject in need thereof a compound, composition or pharmaceutical composition according to the invention.

An aspect relates to a method of treating a patient suffering from a condition selected from diabetes, hyperthyroidism, metabolic syndrome X, or for treating a geriatric patient, which method comprises administering thereto a compound, composition or pharmaceutical composition according to the invention.

Yet a further aspect relates to a method of treating, preventing, or reducing the effects of, neurodegeneration, free radical toxicity, hypoxic conditions or hyperglycemia which method comprises administering to a subject in need thereof a compound, composition or pharmaceutical composition according to the invention. In an embodiment, the neurodegeneration is caused by aging, trauma, anoxia or a neurodegenerative disease or disorder.

An aspect also relates to a method of preventing or treating a neurodegenerative disease or disorder selected from Alzheimer's disease, Parkinson's disease, amyotrophic lateral sclerosis, epilepsy, astrocytoma, glioblastoma and

Huntington's chorea, which method comprises administering to a subject in need thereof a compound, composition or pharmaceutical composition according to the invention.

Another aspect relates to a method of promoting alertness or improving cognitive function in a subject, which method comprises administering to said subject a compound, composition or pharmaceutical composition according to the invention.

An aspect also relates to the use of a compound or composition according to the invention to reduce free fatty acids circulating in the blood plasma of a subject, in the non-therapeutic treatment of muscle impairment or fatigue.

An aspect also relates to the use of a compound or composition according to the invention to non-therapeutically increasing serum concentration of Lac-Phe.

Yet an aspect relates to the use according to the use of a compound or composition according to the invention, wherein the compound or composition is for the non-therapeutic treatment of cardiac muscle fatigue or skeletal muscle fatigue. Yet an aspect relates to the use according to the use of a compound or composition according to the invention, wherein the compound or composition is for the non-therapeutic treatment of heart failure.

The present disclosure relates to a Lactate/Ketone body ester for preservation of vital organ function and combat inflammation and cancer growth. In particular, the present invention relates to a Lactate/Ketone body ester with the beneficial properties of Lactate and beta-hydroxybutyrate (BHB) on vital organ function, inflammation and cancer growth but without the harmful sodium loads following administration of both Lactate and beta-hydroxybutyrate.

The present disclosure relates to Lactate/Ketone body esters for preservation of vital organ function and combat inflammation, cancer growth, sarcopenia and atherosclerosis. In particular, the present invention relates to Lactate/Ketone body esters with the beneficial properties of lactate or lactate and ketone bodies such as beta-hydroxybutyrate (BHB) on vital organ function, inflammation and cancer growth but without harmful ion loads, such as calcium potassium, magnesium and/or sodium loads following administration of both Lactate and beta-hydroxybutyrate salts.

### ITEMS OF THE INVENTION

1. A compound of formula I

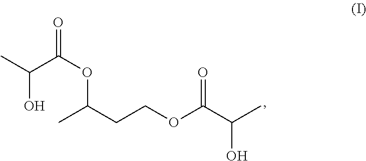 [SMILES: CC(CCOC(=O)C(C)O)OC(=O)C(C)O]

or a pharmaceutical acceptable salt thereof;
or a compound of formula LXII

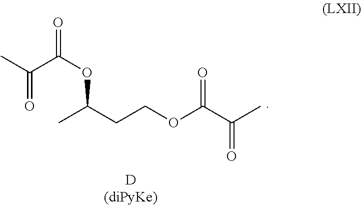 [SMILES: CC(=O)C(=O)OCC[C@@H](C)OC(=O)C(C)=O]

2. The compound according to item 1 with the stereochemistry according to formula II

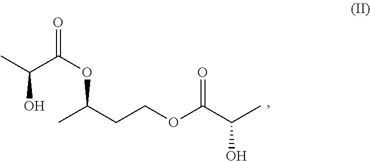 [SMILES: C[C@H](CCOC(=O)[C@H](C)O)OC(=O)[C@H](C)O]

or a pharmaceutical acceptable salt thereof.
3. A composition comprising a compound according to any of the preceding items.
4. The composition according to item 3, comprising a compound according to formula II.
5. The composition according to any of the items 3-4, further comprising one or more compounds selected from the group consisting of:
a compound with the formula X

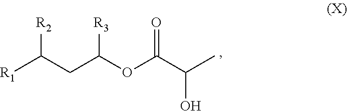

a compound of the formula XI

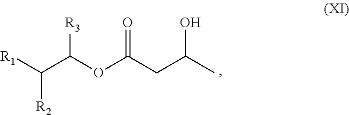

and
a compound of formula XII

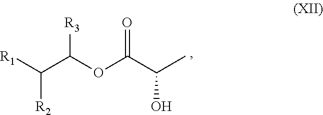

or a pharmaceutical acceptable salt thereof,
wherein R₁, R₂ and R₃ are CH₃, OH and H respectively or R₁, R₂ and R₃ are OH, H and CH₃ respectively.
6. The composition according to any one of the items 3-5, further comprising one or more compounds selected from the group consisting of:
a compound with formula XXII

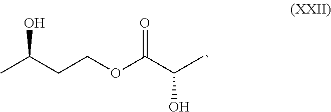

a compound with formula XXIII

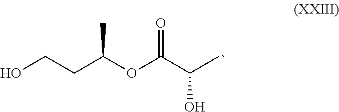

a compound with formula XXIV

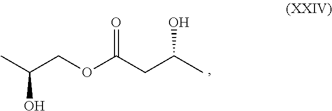

a compound with formula XXV

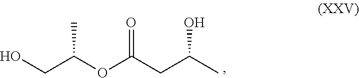

a compound with formula XXVI

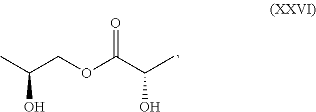

a compound with formula XXVII

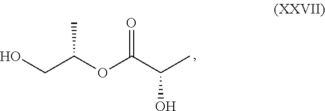

a compound with formula XXVIII

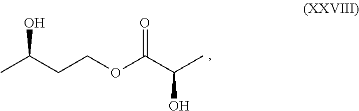

and
a compound with formula XXIX

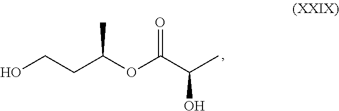

or pharmaceutical acceptable salts thereof.
7. The composition according to any of the items 3-6, comprising a compound of formula II, a compound of formula XXII and/or a compound of formula XXIII.
8. The composition according to any of the items 3-7, comprising a compound of formula II and at least one compound selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.
9. A pharmaceutical composition comprising the compound according to any of items 1-2 or a composition according to any of items 3-8.
10. The compound according to anyone of items 1-2, the composition according to any of items 3-8 or the pharmaceutical composition according to item 9 for use as a medicament.
11. The compound according to any of items 1-2, the composition according to any of items 3-8 or the pharmaceutical composition according to item 9 for use in
   the treatment, prevention or alleviation of inflammatory disease, cancer, epileptic seizures, acute heart failure, Resuscitation, acidosis, traumatic brain injury, acute pancreatitis, hepatitis, myocardial infarction, burns, sepsis, dengue, cognitive dysfunction, atherosclerosis, neurodegeneration, oxidative stress, Alzheimer's disease, Parkinson's disease, amyotrophic lateral sclerosis, epilepsy, astrocytoma, glioblastoma and Huntington's chorea, sarcopenia, muscle atrophy and dysfunctional wound healing;
   the treatment, prevention or alleviation of a condition which is caused by, exacerbated by or associated with elevated plasma levels of free fatty acids in a human or animal subject;
   the treatment, prevention or alleviation of viral infections, immunogenic disorders, non-alcoholic fatty liver disease (NAFLD), non-alcoholic steatohepatitis (NASH), sarcopenia, muscle fatigue, angina pectoris, extreme physical exertion, intermittent claudication, hypoxia, stroke and myocardial infarction, stress, obesity, diabetes, metabolic syndrome, autoimmune disease, muscle impairment or to improve motor function; and/or
   the treatment, prevention and/or alleviation of insulin resistance.
12. A food ingredient comprising the compound according to any of items 1-2 or the composition according to any of items 3-8.
13. A food product comprising the food ingredient according to item 12.
14. The food product according to item 13, wherein the food is selected from the group consisting of a nutraceutical, a food supplement, a dietary supplement, a feed, bar, sugar bar, protein bar, powder, gel, beverage, drink, yoghurt, chewing gum, dairy product, sports drink, confectionary product, ice cream, capsule, tablet, sachet, and pouch.
15. Use of a compound according to any one of items 1-2 or a composition according to any one of items 3-8
   to reduce free fatty acids circulating in the blood plasma of a subject, in the non-therapeutic treatment of muscle impairment, fatigue or improve motor function;
   to suppressing appetite, treating obesity, promoting weight loss, preventing, alleviating and/or treating sarcopenia, maintaining a healthy weight or decreasing the ratio of fat to lean muscle; and/or
   for the non-therapeutic treatment of cardiac muscle fatigue, skeletal muscle fatigue, and/or improvement of motor function or for promoting alertness or improving cognitive function in a subject.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Chemoenzymatic Synthesis of diLaKe

### Aim

To provide a chemoenzymatic synthesis of diLaKe

### Materials and Methods & Results

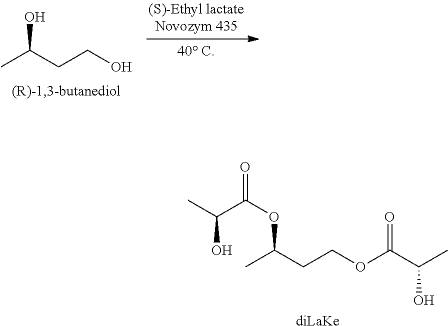

The protocol for the chemoenzymatic synthesis of diLaKe was as follows:

(R)-1,3-Butanediol (0.9 g), (S)-ethyl lactate (11.8 g) and Novozym 435 (0.5 g) was placed in a sealed vial and gently vortexed at 40° C. for 40 h. At this time, GC-analysis revealed 90% conversion of (R)-1,3-butanediol and 20% diLaKe. Following removal of excess (S)-ethyl lactate by distillation, diLaKe could be obtained at analytical purity by flash column chromatography on silica gel.

CalB may be used instead of Novozym 435 in the synthesis. This resulted in 10% diLaKe as determined by GC-analysis after 40 h.

### 1H NMR

diLaKe: (400 MHZ, CDCl₃) δ_{H} 5.11 (m, 1H), 4.24 (m, 4H), 1.97 (m, 2H), 1.41 (m, 6H), 1.30 (m, 3H)

Using NMR spectroscopy, it was determined that diLaKe was produced as outlined above.

### Conclusion

diLaKe may be produced via chemoenzymatic synthesis

### Example 2—Chemical Synthesis of diLaKe

### Aim

To provide a chemical synthesis of diLaKe

### Materials and Methods

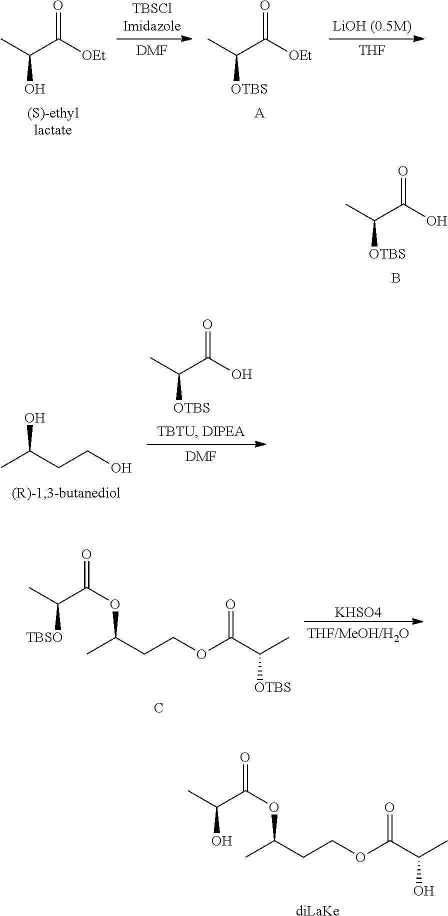

The protocol for the synthesis of diLaKe is as follows:

To a solution of (S)-ethyl lactate (14.4 mL, 127 mmol, 1.0 eq.) in anhydrous DMF (250 mL) was added TBSCl (28.7 g, 191 mmol, 1.5 eq.) followed by imidazole (30.3 g, 445 mmol, 3.5 eq.). The mixture was stirred at r.t. for 3 hours before diluted with sat. aq. NaCl and extracted with EtOAc. The organic layers were combined and washed with 5% aq. HCl and sat. aq. NaCl. The organic layer was then dried over Na₂SO₄, filtered and concentrated to afford ethyl(S)-2-(tert-butyldimethylsilyloxy) propanoate A (29.5 g, quant.). Product formation was determined with 1H NMR analysis. No further purification was performed as the product was subjected directly into the following step.

### R_{f}

0.41 (Pentane/EtOAc 98:2, KMnO₄).

### ¹H NMR

(400 MHZ, CDCl₃) δ_{H} 4.34-4.26 (q, J=6.74 Hz, 1H), 4.24-4.11 (m, 2H), 1.41-1.36 (d, J=6.75 Hz, 3H), 1.30-1.24 (t, J=7.16 Hz, 3H), 0.91-0.87 (s, 9H), 0.11-0.04 (d, J=11.99 Hz, 6H).

To a solution of ethyl(S)-2-(tert-butyldimethylsilyloxy) propanoate A (29.5 g, 127 mmol, 1.0 eq.) in THF (400 mL) at 0° C. was added a cooled aq. LiOH solution (508 mL, 0.5 M). The reaction mixture was stirred at r.t. for 5 hours before concentrated to half of the original volume and extracted with Et₂O. The organic extracts were combined before extracted with a sat. aq. NaHCO₃ solution. The aqueous layers were combined and acidified with a 1 M aq. KHSO₄ solution to reach pH≈3-4. Afterwards, the aqueous solution was extracted thoroughly with Et₂O and the organic layers were combined, dried over Na₂SO₄ and concentrated to afford (S)-2-(tert-butyldimethylsilyloxy) propanoic acid B (22.3 g, 86%) as an oil. Product formation was determined with ¹H NMR analysis (¹H NMR values are in accordance with reported values) (10).

### ¹H NMR

(400 MHZ, CDCl₃) δ_{H} 4.38-4.33 (q, J=6.88 Hz, 1H), 1.46-1.45 (d, J=6.80 Hz, 3H), 0.94-0.90 (m, 9H), 0.16-0.12 (s, 6H).

(S)-2-(tert-butyldimethylsilyloxy) propanoic acid B (2.1 equiv.), TBTU (3.0 equiv.) and DIPEA (4.2 equiv.) is dissolved in anhydrous DMF and the mixture stirred at r.t. for 1 hour. (R)-(−)-1,3-butanediol (1.0 equiv.) in anhydrous DMF is added and the reaction mixture stirred o/n. at ambient temperature. The reaction mixture is diluted with CH₂Cl₂ and the resulting mixture washed with 1 M aq. HCl, aq. NaHCO₃ and water sequentially. The organic layers is then combined, dried over Na₂SO₄, filtered and concentrated to afford the crude product. Flash column chromatography on silica gel (or an alternative purification method) is performed to afford the pure product (C)

To a stirred solution of C (1.0 equiv.) in THF:H₂O:MeOH (2.5:2.5:1) is added KHSO₄ (0.25 equiv.) and the mixture stirred at r.t. o/n. MeOH is removed under reduced pressure and water added before extraction with EtOAc. The organic layers is collected and dried over Na₂SO₄, filtered and concentrated. Flash column chromatography on silica gel (or an alternative purification method) is performed to afford the pure product diLaKe.

### Results

Using NMR spectroscopy, it will be determined that diLaKe is produced as outlined above.

### Conclusion

diLaKe will be produced via chemical synthesis

### Example 3—Design of Animal Experiment

### Preparation of the Drug

The diLaKe ester (formula II) is diluted in physiological saline solution (0.9% NaCl) and administered as a single bolus of 2 mL, per oral dose. The dose of 4500 mg/kg is based on a previous pilot series. Placebo animals receive a single bolus of 2 mL physiological saline solution (0.9% NaCl).

### Animal Setup:

Male Sprague Dawley rats are kept for acclimatization at a constant temperature of 23° C., with a 12 h light-dark cycle and with unlimited access to food and water. All animal handling is in accordance with national guidelines in Denmark and the Guide for the Care and Use of Laboratory Animals¹ and all experiments conformed to Danish Law (Act. No. 1306 of 23 Nov. 2007).

The rats are randomly selected to receive either diLaKe or placebo.

Rats are anaesthetised in an induction chamber with 8% Sevoflurane (Sevorane®, AbbVIE A/S, Copenhagen, Denmark) mixed with oxygen saturated atmospheric air (flow: 2.0 L/min).

Upon achieved anaesthesia, the rats are intubated and connected to a mechanical ventilator (Ugo Basile 7025 rodent ventilator, Comerio, Varese, Italy) with an adjusted flow of 1.0 L/min with 3.5% Sevoflurane. Body temperature is kept at a constant 37° C.±1° C. with a temperature probe (UNO, Zevenaar, Holland). A PTFE coated flexible orogastric tube (Fuchigami, Japan) is placed and the rat is left for stabilization for 15 minutes.

After stabilization a baseline blood sample is collected from the rat tail vein before administration of diLaKe or placebo.

Following the baseline blood sample, a bolus of diLaKe solution or placebo is administered via the orogastric tube to the animals. Every 15 minutes for a period of two hours, blood samples (200 uL each) are collected in microvettes (sarstedt-20.1280.100) and is left to coagulate for 30 minutes followed by centrifugation at 4° C., 1500 G for 20 minutes. Serum is collected and stored at −80° C. for further analysis.

### Example 4—Quantification of BHB and Lactate in Rat Serum-diLaKe (Formula II)

The concentration of BHB and lactate will be determined in the blood samples isolated from the rats according to example 3. Rat serum isolated every 15 minutes for a 2 hours are analyzed. The quantification is done on LC-MS/MS using isotopically labeled internal standards (7).

### Conclusion

Here we will show that treatment with diLaKe (formula II), compared to the control (physiological saline 0.9%) gives rise to an increased serum concentration of both BHB and lactate in rats following oral administration, similar to the increased serum concentrations after treatment with LaKe as demonstrated in Example 10.

### Example 5—Effect of the diLaKe Ester (Formula II) on Free Fatty Acids

The concentration of free-fatty acids (FFA) is determined in the blood samples isolated from the rats according to example 3. The level of FFA in blood reflects ongoing lipolysis and high levels of FFA induce insulin resistance; conversely agents that lower the level of free fatty acids in the blood can be used therapeutically to increase insulin sensitivity in people with type 2 diabetes and the metabolic syndrome.

Concentrations of non-esterified free fatty acids (NEFA) are measured using a NEFA-HR (2) kit (Wako, Chemicals Gmbh). Absorbance is measured by spectrometry (PHERAstar FS, BMG LABTECH, Ortenberg, Germany).

The concentration (mM) of free fatty acids in the serum is expected to be decreased in the rats treated with diLaKe compared to rats treated with the control (physiological saline 0.9%).

### Conclusion

Here, we will show that oral administration of diLaKe (formula II) leads to a decreased concentration of FFA in rat serum compared to the control, similar to the decreased concentration after treatment with LaKe as demonstrated in Example 11.

### Example 6—Chemical Synthesis of the LaKe Compounds 3 and 4 (Formula XXII and XXIII)

The LaKe compounds are prepared according to the following procedure.

### Synthesis of the Compounds XXII and XXIII:

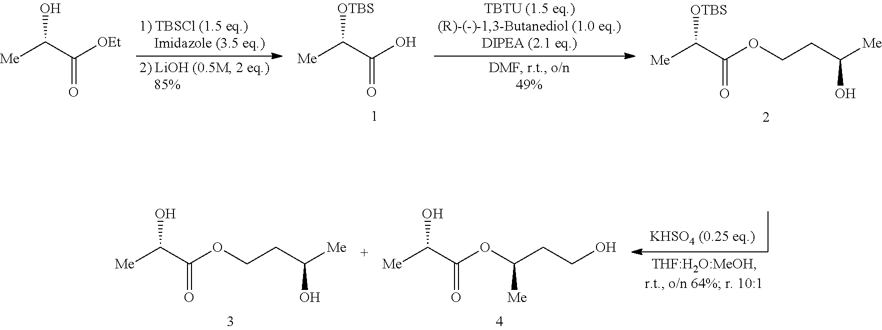 [SMILES: CCOC(=O)[C@H](C)O ; C[C@H](O[Si](C)(C)C(C)(C)C)C(=O)O ; C[C@H](O[Si](C)(C)C(C)(C)C)C(=O)OCC[C@@H](C)O ; C[C@H](O)C(=O)OCC[C@@H](C)O ; C[C@H](CCO)OC(=O)[C@H](C)O]

### (S)-2-((tert-butyldimethylsilyl)oxy) propanoic acid (1)

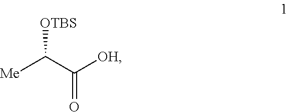

To a solution of ethyl(S)-2-hydroxypropionate (14.4 mL, 127 mmol, 1.0 eq.) in anhydrous DMF (250 mL) was added TBSCl (28.7 g, 191 mmol, 1.5 eq.) followed by imidazole (30.3 g, 445 mmol, 3.5 eq.). The mixture was stirred at r.t. for 3 hours before diluted with sat. aq. NaCl and extracted with EtOAc. The organic layers were combined and washed with 5% aq. HCl and sat. aq. NaCl. The organic layer was then dried over Na₂SO₄, filtered and concentrated to afford ethyl(S)-2-(tert-butyldimethylsilyloxy) propanoate (29.5 g, quant.). Product formation was determined with 1H NMR analysis. No further purification was performed as the product was subjected directly into the following step.

### R_{f}

0.41 (Pentane/EtOAc 98:2, KMnO₄).

### ¹H NMR

(400 MHZ, CDCl₃) δ_{H} 4.34-4.26 (q, J=6.74 Hz, 1H), 4.24-4.11 (m, 2H), 1.41-1.36 (d, J=6.75 Hz, 3H), 1.30-1.24 (t, J=7.16 Hz, 3H), 0.91-0.87 (s, 9H), 0.11-0.04 (d, J=11.99 Hz, 6H).

To a solution of(S)-2-(tert-butyldimethylsilyloxy) propanoate (29.5 g, 127 mmol, 1.0 eq.) in THF (400 mL) at 0° C. was added a cooled aq. LiOH solution (508 mL, 0.5 M). The reaction mixture was stirred at r.t. for 5 hours before concentrated to half of the original volume and extracted with Et₂O. The organic extracts were combined before extracted with a sat. aq. NaHCO₃ solution. The aqueous layers were combined and acidified with a 1 M aq. KHSO₄ solution to reach pH≈3-4. Afterwards, the aqueous solution was extracted thoroughly with Et₂O and the organic layers were combined, dried over Na₂SO₄ and concentrated to afford(S)-2-(tert-butyldimethylsilyloxy) propanoic acid (22.3 g, 86%) as an oil. Product formation was determined with ¹H NMR analysis (¹H NMR values are in accordance with reported values) (10).

### ¹H NMR

(400 MHZ, CDCl₃) δ_{H} 4.38-4.33 (q, J=6.88 Hz, 1H), 1.46-1.45 (d, J=6.80 Hz, 3H), 0.94-0.90 (m, 9H), 0.16-0.12 (s, 6H).

### (R)-3-Hydroxybutyl(S)-2-(((tert)-butyldimethylsilyl)oxy) propanoate (2)

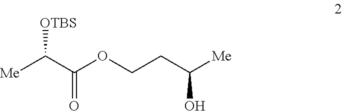 [SMILES: C[C@H](O[Si](C)(C)C(C)(C)C)C(=O)OCC[C@@H](C)O]

(S)-2-(tert-butyldimethylsilyloxy) propanoic acid (8.42 g, 41.2 mmol, 1.0 eq.), TBTU (19.9 g, 61.8 mmol, 1.5 eq.) and DIPEA (15.1 mL, 86.5 mmol, 2.1 eq.) were dissolved in anhydrous DMF (150 mL) and the mixture was stirred at r.t. for 1 hour. (R)-(−)-1,3-butanediol (3.68 mL, 41.2 mmol, 1.0 eq.) in anhydrous DMF (10 mL) was then added and the reaction mixture was stirred o/n. at ambient temperature. The reaction mixture was diluted with CH₂Cl₂ and the resulting mixture was washed with 1 M aq. HCl, aq. NaHCO₃ and water sequentially. The organic layers were then combined, dried over Na₂SO₄, filtered and concentrated to afford the crude product. FCC (pentane/EtOAc=95:5 to 85:15) was performed to afford the pure product (2) (5601 mg, 49%).

### R_{f}

0.38 (Pentane/EtOAc 8:2, KMnO₄).

### ¹H NMR

(400 MHZ, CDCl₃) δ_{H} 4.42-4.30 (m, 2H), 4.21-4.16 (m, 1H), 3.93-3.84 (m, 1H), 2.00-1.97 (d, J=4.27 Hz, 1H), 1.85-1.69 (m, 2H), 1.42-1.38 (d, J=6.79 Hz, 3H), 1.24-1.21 (d, J=6.27 Hz, 3H), 0.91-0.89 (s, 9H), 0.11-0.07 (d, J=10.17 Hz, 6H).

### ¹³C NMR

(101 MHz, CDCl₃) δ_{C} 174.6, 68.6, 65.1, 62.3, 38.2, 25.9, 23.6, 21.5, 18.4, −4.8, −5.1.

### IR (Neat)

Uₘₐₓ/cm⁻¹ 3440, 2930, 2858, 1737, 1463, 1373, 1256, 1140.

### HRMS

[M+Na]⁺=299.1649; found: 299.1644.
[α]^{24.0}_{D}

−34.6° (C=10 mg/mL, CHCl₃).

### (R)-3-Hydroxybutyl(S)-2-hydroxypropanoate (3) and (R)-4-hydroxybutan-2-yl (S)-2-hydroxypropanoate (4)

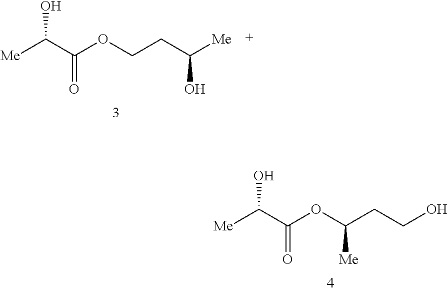 [SMILES: C[C@H](O)C(=O)OCC[C@@H](C)O ; C[C@H](CCO)OC(=O)[C@H](C)O]

To a stirred solution of TBDMS ether (5612 mg, 20.3 mmol, 1.0 eq.) in THF:H₂O:MeOH (2.5:2.5:1, 100 mL) was added KHSO₄ (691 mg, 5.08 mmol, 0.25 eq.) and the mixture was stirred at r.t. o/n. MeOH was removed under reduced pressure and water was added before extracted with EtOAc. The organic layers were collected and dried over Na₂SO₄, filtered and concentrated. The crude product was purified with FCC (Pentane/EtOAc=1:1) to afford the desired products (2100 mg, 68%, r. 10:1).

### R_{f}

0.19 (Pentane/EtOAc 1:1, CAM).

### ¹H NMR

3: (400 MHZ, CDCl₃) δ_{H} 4.46-4.37 (m, 1H), 4.32-4.23 (m, 2H), 3.95-3.85 (m, 1H), 2.81-2.79 (d, J=5.28 Hz, 1H), 1.87-1.71 (m, 3H), 1.44-1.39 (d, J=6.97 Hz, 3H), 1.27-1.22 (d, J=6.53 Hz, 3H).

4: (400 MHZ, CDCl₃) δ_{H} 5.25-5.17 (m, 1H), 4.46-4.37 (m, 1H), 3.74-3.57 (m, 2H), 1.87-1.71 (m, 2H), 1.44-1.39 (d, J=6.97 Hz, 3H), 1.33-1.30 (d, J=6.22 Hz, 3H).

### ¹³C NMR

3: (101 MHz, CDCl₃) δ_{C} 176.1, 66.9, 64.9, 63.0, 38.0, 23.8, 20.6.

### IR (Neat)

υₘₐₓ/cm⁻¹ 3380, 2971, 1732, 1457, 1375, 1274, 1209, 1130, 1049.

### HRMS

[M+Na]⁺=185.0784; found: 185.0783.

### Example 7—Chemical Synthesis of the LaKe Compounds 10 and 11 (Formula XXVIII and XXIX)

The LaKe compounds are prepared according to the following procedure.

### Synthesis of the Compounds XXVIII and XXIX:

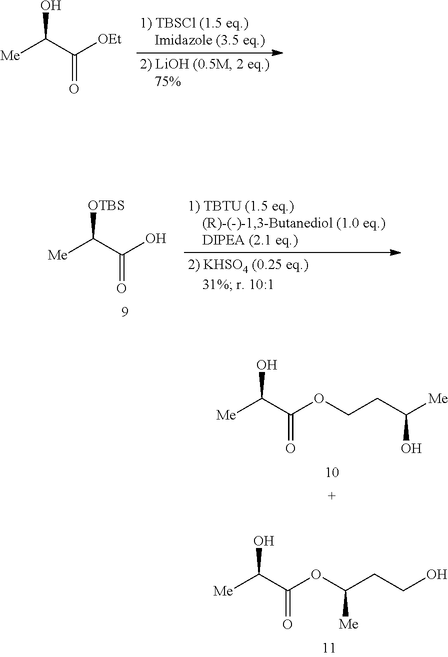 [SMILES: CCOC(=O)[C@@H](C)O ; C[C@@H](O[Si](C)(C)C(C)(C)C)C(=O)O ; C[C@@H](O)CCOC(=O)[C@@H](C)O ; C[C@H](CCO)OC(=O)[C@@H](C)O]

### (R)-2-((tert-butyldimethylsilyl)oxy) propanoic acid (9)

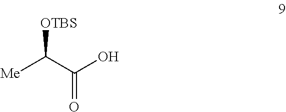

To a solution of ethyl (R)-2-hydroxypropionate (6.74 mL, 59.3 mmol, 1.0 eq.) in anhydrous DMF (100 mL) was added TBSCl (13.4 g, 89 mmol, 1.5 eq.) followed by imidazole (14.1 g, 208 mmol, 3.5 eq.). The mixture was stirred at r.t. for 3 hours before diluted with sat. aq. NaCl and extracted with EtOAc. The organic layers were combined and washed with 5% aq. HCl and sat. aq. NaCl. The organic layer was then dried over Na₂SO₄, filtered and concentrated to afford ethyl (R)-2-(tert-butyldimethylsilyloxy) propanoate (13.7 g, quant.). Product formation was determined with 1H NMR analysis. No further purification was performed as the product was subjected directly into the following step.

### R_{f}

0.41 (Pentane/EtOAc 98:2, KMnO₄).

### ¹H NMR

(400 MHZ, CDCl₃) δ_{H} 4.34-4.26 (q, J=6.74 Hz, 1H), 4.24-4.11 (m, 2H), 1.41-1.36 (d, J=6.75 Hz, 3H), 1.30-1.24 (t, J=7.16 Hz, 3H), 0.91-0.87 (s, 9H), 0.11-0.04 (d, J=11.99 Hz, 6H).

To a solution of the (R)-2-(tert-butyldimethylsilyloxy) propanoate (13.7 g, 59.3 mmol, 1.0 eq.) in THF (400 mL) at 0° C. was added a cooled aq. LiOH solution (400 mL, 0.3 M). The reaction mixture was stirred at r.t. for 5 hours before concentrated to half of the original volume and extracted with Et₂O. The organic extracts were combined before extracted with a sat. aq. NaHCO₃ solution. The aqueous layers were combined and acidified with a 1 M aq. KHSO₄ solution to reach pH≈3-4. Afterwards, the aqueous solution was extracted thoroughly with Et₂O and the organic layers were combined, dried over Na₂SO₄ and concentrated to afford (R)-2-(tert-butyldimethylsilyloxy) propanoic acid (9085 mg, 75%) as an oil. Product formation was determined with 1H NMR analysis (1H NMR values are in accordance with reported values) 1.

### ¹H NMR

(400 MHZ, CDCl₃) δ_{H} 4.38-4.33 (q, J=6.88 Hz, 1H), 1.46-1.45 (d, J=6.80 Hz, 3H), 0.94-0.90 (m, 9H), 0.16-0.12 (s, 6H).

### (R)-3-Hydroxybutyl (R)-2-hydroxypropanoate (10) and (R)-4-hydroxybutan-2-yl (R)-2-hydroxypropanoate (11)

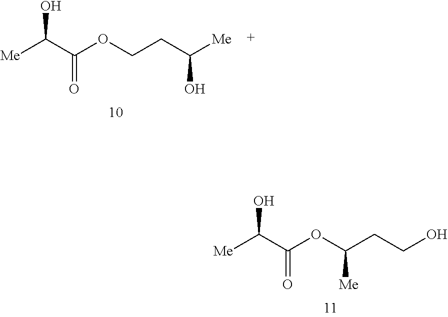 [SMILES: C[C@@H](O)CCOC(=O)[C@@H](C)O ; C[C@H](CCO)OC(=O)[C@@H](C)O]

(R)-2-((tert-butyldimethylsilyl)oxy) propanoic acid (8.99 g, 44.0 mmol, 1.0 eq.), TBTU (21.3 g, 66.0 mmol, 1.5 eq.) and DIPEA (16.1 mL, 92.4 mmol, 2.1 eq.) were dissolved in anhydrous DMF (175 mL) and the mixture was stirred at r.t. for 1 hour. (R)-(−)-1,3-butanediol (3.93 mL, 44.0 mmol, 1.0 eq.) in anhydrous DMF (10 mL) was then added and the reaction mixture was stirred o/n. at ambient temperature. The reaction mixture was diluted with CH₂Cl₂ and the resulting mixture was washed with 1 M aq. HCl, aq. NaHCO₃ and water sequentially. The organic layers were then combined, dried over Na₂SO₄, filtered and concentrated to afford the crude product. FCC (pentane/EtOAc=95:5 to 85:15) was performed to afford the pure product (5576 mg, 46%).

### R_{f}

0.38 (Pentane/EtOAc 8:2, KMnO₄).

### ¹H NMR

(400 MHZ, CDCl₃) δ_{H} 4.44-4.36 (m, 1H), 4.36-4.29 (q, J=6.58 Hz, 1H), 4.20-4.13 (m, 1H), 3.93-3.82 (m, 1H), 2.01-1.96 (d, J=4.44 Hz, 1H), 1.86-1.67 (m, 2H), 1.42-1.38 (d, J=6.69 Hz, 3H), 1.25-1.20 (d, J=6.21 Hz, 3H), 0.93-0.87 (s, 9H), 0.11-0.07 (d, J=10.35 Hz, 6H).

### IR (Neat)

Uₘₐₓ/cm⁻¹ 3449, 2958, 2930, 2900, 2858, 1736, 1253, 1138.

### HRMS

[M+Na]⁺=299.1649; found: 299.1658.

[α]^{24.0}_{D}

+17.1° (C=8.5 mg/mL, CHCl₃).

To a stirred solution of TBDMS ether (5576 mg, 20.2 mmol, 1.0 eq.) in THF:H₂O:MeOH (2.5:2.5:1, 100 mL) was added KHSO₄ (668 mg, 5.05 mmol, 0.25 eq.) and the mixture was stirred at r.t. o/n. MeOH was removed under reduced pressure and water was added before extracted with EtOAc. The organic layers were collected and dried over Na₂SO₄, filtered and concentrated. The crude product was purified with FCC (Pentane/EtOAc=1:1) to afford the desired products (2110 mg, 64%, r. 10:1).

### R_{f}

0.19 (Pentane/EtOAc 1:1, CAM).

### ¹H NMR

10: (400 MHZ, CDCl₃) δ_{H} 4.49-4.38 (m, 1H), 4.33-4.20 (m, 2H), 3.95-3.83 (m, 1H), 2.82-2.74 (d, J=4.96 Hz, 1H), 1.98-1.61 (m, 3H), 1.44-1.39 (d, J=6.92 Hz, 3H), 1.27-1.22 (d, J=6.22 Hz, 3H).

11: (400 MHZ, CDCl₃) δ_{H} 5.25-5.17 (m, 1H), 4.49-4.38 (m, 1H), 3.71-3.64 (m, 1H), 3.63-3.54 (m, 1H), 1.98-1.61 (m, 3H), 1.44-1.39 (d, J=6.92 Hz, 3H), 1.34-1.20 (d, J=6.21 Hz, 3H).

### ¹³C NMR

10: (101 MHZ, CDCl₃) δ_{C} 176.1, 66.9, 64.9, 63.0, 37.9, 23.7, 20.5.

### IR (Neat)

Uₘₐₓ/cm⁻¹ 3377, 2971, 2901, 1732, 1376, 1209, 1128, 1079, 1049.

### HRMS

[M+Na]⁺=185.0784; found: 185.0790.

### Example 8—Synthesis of the DiLa Compounds 7 and 8 (Formula XXVI and XXVII)

### Synthesis of the Compounds XXVI and XXVII:

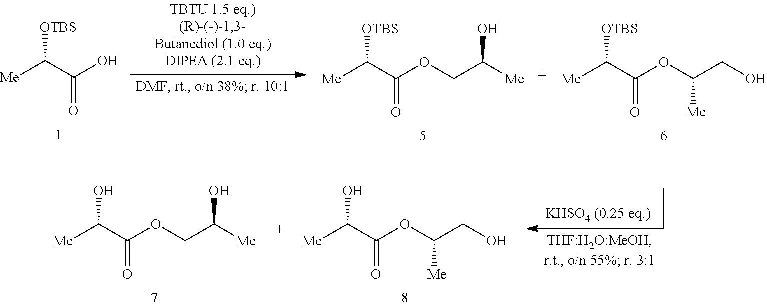 [SMILES: C[C@H](O[Si](C)(C)C(C)(C)C)C(=O)O ; C[C@H](O)COC(=O)[C@H](C)O[Si](C)(C)C(C)(C)C ; C[C@H](O[Si](C)(C)C(C)(C)C)C(=O)O[C@@H](C)CO ; C[C@H](O)COC(=O)[C@H](C)O ; C[C@H](O)C(=O)O[C@@H](C)CO]

### (S)-2-Hydroxypropyl(S)-2-((tert-butyldimethylsilyl)oxy) propanoate (5) and(S)-1-hydroxypropan-2-yl(S)-2-((tert-butyldimethylsilyl)oxy) propanoate (6)

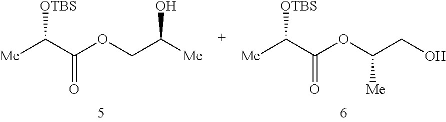 [SMILES: C[C@H](O)COC(=O)[C@H](C)O[Si](C)(C)C(C)(C)C ; C[C@H](O[Si](C)(C)C(C)(C)C)C(=O)O[C@@H](C)CO]

(S)-2-(tert-butyldimethylsilyloxy) propanoic acid (5.80 g, 28.4 mmol, 1.0 eq.), TBTU (13.7 g, 42.6 mmol, 1.5 eq.) and DIPEA (10.4 mL, 59.6 mmol, 2.1 eq.) were dissolved in anhydrous DMF (80 mL) and the mixture was stirred at r.t. for 1 hour. (S)-(−)-1,2-propanediol (2.08 mL, 28.4 mmol, 1.0 eq.) in anhydrous DMF (10 mL) was then added and the reaction mixture was stirred o/n. at ambient temperature. The reaction mixture was diluted with CH₂Cl₂ and the resulting mixture was washed with 1 M aq. HCl, aq. NaHCO₃ and water sequentially. The organic layers were then combined, dried over Na₂SO₄, filtered and concentrated to afford the crude product. FCC (pentane/EtOAc=85:15) was performed to afford 5 and 6 (2850 mg, 38%; r. 10:1).

### R_{f}

0.14 (Pentane/EtOAc 9:1, KMnO₄).

### ¹H NMR

5: (400 MHZ, CDCl₃) δ_{H} 4.40-4.32 (q, J=6.79 Hz, 1H), 4.16-4.10 (dd, J=10.79 Hz, 2.76 Hz, 1H), 4.08-4.01 (m, 1H), 4.01-3.94 (dd, J=10.77 Hz, 7.13 Hz, 1H), 2.14-2.09 (d, J=3.47 Hz, 1H), 1.44-1.39 (d, J=6.72 Hz, 3H), 1.23-1.18 (d, J=6.26 Hz, 3H), 0.92-0.88 (s, 9H), 0.12-0.05 (d, J=9.51 Hz, 6H).

6: (400 MHZ, CDCl₃) δ_{H} 5.05-4.96 (m, 1H), 4.34-4.29 (q, J=6.69 Hz, 1H), 3.71-3.58 (m, 2H), 1.96-1.91 (t, J=6.05 Hz, 1H), 1.41-1.38 (d, J=6.72 Hz, 3H), 1.26-1.23 (d, J=6.56 Hz, 3H), 0.92-0.88 (s, 9H), 0.12-0.05 (d, J=9.51 Hz, 6H).

### ¹³C NMR

5: (101 MHZ, CDCl₃) δ_{C} 174.3, 70.0, 68.5, 66.2, 25.8, 21.4, 19.2, 18.4, −4.8, −5.1.

### IR (Neat)

Uₘₐₓ/cm⁻¹ 3508, 2953, 2931, 2887, 2857, 1739, 1472, 1254, 1203, 1141, 1061.

### HRMS

[M+Na]⁺=285.1493; found: 285.1497.

### (S)-2-Hydroxypropyl(S)-2-hydroxypropanoate (7) and (S)-1-hydroxypropan-2-yl (S)-2-hydroxypropanoate (8)

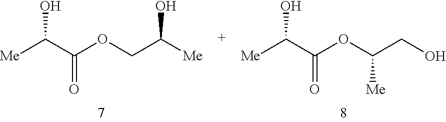 [SMILES: C[C@H](O)COC(=O)[C@H](C)O ; C[C@H](O)C(=O)O[C@@H](C)CO]

To a stirred solution of TBDMS ether (2834 mg, 10.8 mmol, 1.0 eq.) in THF:H₂O:MeOH (2.5:2.5:1, 50 mL) was added KHSO₄ (368 mg, 2.70 mmol, 0.25 eq.) and the mixture was stirred at r.t. o/n. MeOH was removed under reduced pressure and water was added before extracted with EtOAc. The organic layers were collected and dried over Na₂SO₄, filtered and concentrated. The crude product was purified with FCC (Pentane/EtOAc=1:1 to 4:6) to afford the desired products (880 mg, 55%, r. 3:1).

### R_{f}

0.12 (Pentane/EtOAc 1:1, KMnO₄).

### ¹H NMR

7: (400 MHZ, CDCl₃) δ_{H} 4.37-4.29 (q, J=6.62 Hz, 1H), 4.18-4.12 (dd, J=10.70 Hz, 2.54 Hz, 1H), 4.08-4.00 (m, 1H), 4.00-3.94 (dd, J=10.71 Hz, 7.28 Hz, 1H), 3.90-3.83 (s, 1H), 3.38-3.30 (s, 1H), 1.44-1.40 (d, J=6.99 Hz, 3H), 1.21-1.17 (d, J=6.14 Hz, 3H).

8: (400 MHZ, CDCl₃) δ_{H} 5.06-4.97 (m, 1H), 4.30-4.24 (q, J=6.81 Hz, 1H), 3.86-3.80 (m, 1H), 3.70-3.63 (d, J=11.81 Hz, 2H), 3.63-3.53 (m, 1H), 3.28-3.19 (s, 1H), 1.42 1.38 (d, J=6.72 Hz, 3H), 1.25-1.21 (d, J=6.43 Hz, 3H).

### ¹³C NMR

7: (101 MHZ, CDCl₃) δ_{C} 175.3, 70.2, 67.2, 65.8, 20.4, 19.0.

8: (101 MHZ, CDCl₃) δ_{C} 175.3, 73.1, 67.4, 65.3, 20.4, 16.1.

### IR (Neat)

Uₘₐₓ/cm⁻¹ 3391, 2984, 1734, 1455, 1374, 1213, 1130, 1054.

### HRMS

[M+Na]⁺=171.0628; found: 171.0629.

### Example 9—Design of Animal Experiment

### Preparation of the Drug

The LaKe ester (formula XXII and XXIII) was diluted in physiological saline solution (0.9% NaCl) and administered as a single bolus of 2 mL, per oral dose. The dose of 4500 mg/kg was based on a previous pilot series (data not shown).

Placebo animals received a single bolus of 2 mL physiological saline solution (0.9% NaCl).

### Animal Setup:

Male Sprague Dawley rats (300-350 g, Taconic, Ry, Denmark) were kept for acclimatization at a constant temperature of 23° C., with a 12 h light-dark cycle and with unlimited access to food and water. All animal handling was in accordance with national guidelines in Denmark and the Guide for the Care and Use of Laboratory Animals¹ and all experiments conformed to Danish Law (Act. No. 1306 of 23 Nov. 2007).

The rats were randomly selected to receive either LaKe (n=8) or placebo (n=8).

Rats were anaesthetised in an induction chamber with 8% Sevoflurane (Sevorane®, AbbVIE A/S, Copenhagen, Denmark) mixed with oxygen saturated atmospheric air (flow: 2.0 L/min).

Upon achieved anaesthesia, the rats were intubated and connected to a mechanical ventilator (Ugo Basile 7025 rodent ventilator, Comerio, Varese, Italy) with an adjusted flow of 1.0 L/min with 3.5% Sevoflurane. Body temperature was kept at a constant 37° C.±1° C. with a temperature probe (UNO, Zevenaar, Holland). A PTFE coated flexible orogastric tube (Fuchigami, Japan) was placed and the rat was left for stabilization for 15 minutes.

After stabilization a baseline blood sample was collected from the rat tail vein before administration of LaKe or placebo.

Following the baseline blood sample, a bolus of LaKe solution or placebo was administered via the orogastric tube to the animals. Every 15 minutes for a period of two hours, blood samples (200 μL each) were collected in microvettes (sarstedt-20.1280.100) and was left to coagulate for 30 minutes followed by centrifugation at 4° C., 1500 G for 20 minutes. Serum was collected and stored at −80° C. for further analysis.

### Statistical Analysis

Based on own experience and reports by other research groups, a sample size of n=8 was considered adequate to identify a treatment effect. All results are expressed as mean±SD unless otherwise stated. Differences in concentrations were analysed using two-way ANOVA with Bonferroni post hoc correction. All analyses were performed using GraphPad Prism 8.2.0 (Graph Pad Software, CA, USA). P<0.05 was considered statistically significant.

### Example 10—Quantification of BHB and Lactate in Rat Serum-LaKe (formula XXII and XXIII)

The concentration of BHB and lactate is determined in the blood samples isolated from the rats according to example 4. Rat serum isolated every 15 minutes for a 2 hours were analyzed. The quantification was done on LC-MS/MS using isotopically labeled internal standards (7).

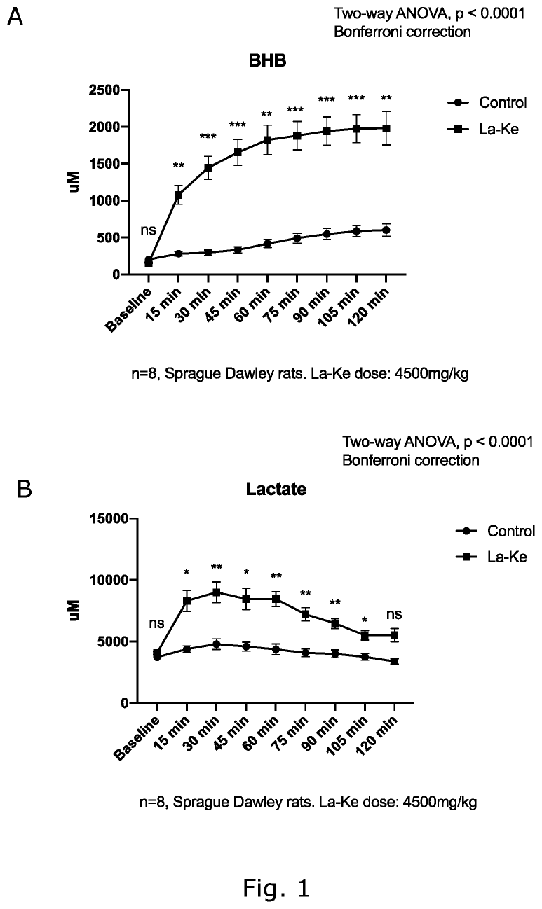

FIG. 1A: BHB concentration (UM) in rat serum isolated from two groups of rats, one treated with Lake, one treated with the control.

FIG. 1B: Lactate concentration (UM) in rat serum isolated from two groups of rats, one treated with Lake, one treated with the control.

### Conclusion

Here we show that treatment with LaKe (formula XXII and XXIII), compared to the control (physiological saline 0.9%) gives rise to an increased serum concentration of both BHB and lactate in rats following oral administration.

### Example 11—Effect of the LaKe Ester (Formula XXII and XXIII) on Free Fatty Acids

The concentration of free-fatty acids (FFA) was determined in the blood samples isolated from the rats according to example 4. The level of FFA in blood reflects ongoing lipolysis and high levels of FFA induce insulin resistance; conversely agents that lower the level of free fatty acids in the blood can be used therapeutically to increase insulin sensitivity in people with type 2 diabetes and the metabolic syndrome.

Concentrations of non-esterified free fatty acids (NEFA) were measured using a NEFA-HR (2) kit (Wako, Chemicals Gmbh). Absorbance was measured by spectrometry (PHERAstar FS, BMG LABTECH, Ortenberg, Germany).

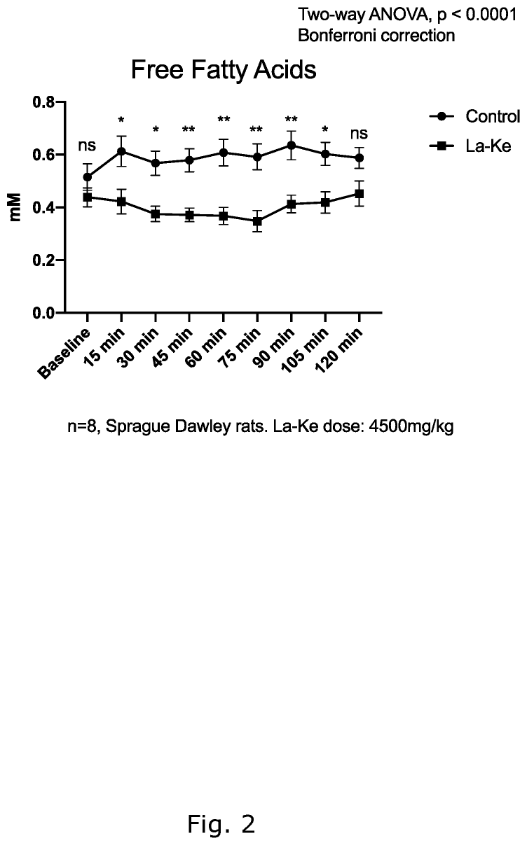

FIG. 2: The concentration (mM) of free fatty acids in the serum was strongly decreased in the rats treated with LaKe compared to rats treated with the control (physiological saline 0.9%).

### Conclusion

Here, we show that oral administration of LaKe (formula XXII and XXIII) leads to a decreased concentration of FFA in rat serum compared to the control.

### Example 12—Quantification of BHB and Lactate in Rat Serum-KeLa (Formula XXIV and XXV)

The concentration of BHB and lactate is determined in the blood samples isolated from the rats according to example 4, with the difference that KeLa (formula XXIV and XXV) was used. Rat serum isolated every 15 minutes for a 2 hours were analyzed. The quantification was done on LC-MS/MS using isotopically labeled internal standards (7).

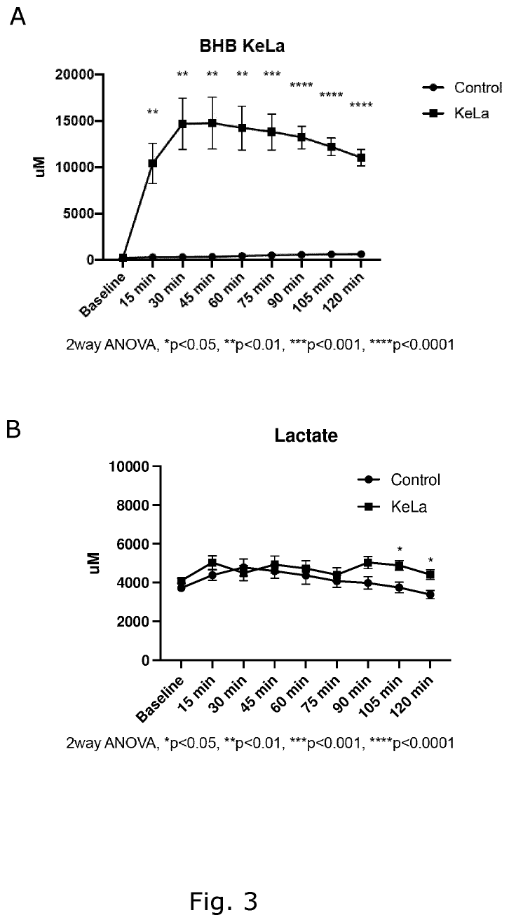

FIG. 3A: BHB concentration (μM) in rat serum isolated from two groups of rats, one treated with KeLa, one treated with the control.

FIG. 3B: Lactate concentration (μM) in rat serum isolated from two groups of rats, one treated with KeLa, one treated with the control. The increase in lactate for the KeLa ester is statistically significant from 90 min and onwards.

### Conclusion

Here we show that treatment with KeLa (formula XXIV and XXV) compared to the control (physiological saline 0.9%) gives rise to an increased serum concentration of both BHB and lactate in rats following oral administration.

### Example 13—Quantification of BHB and Lactate in Rat Serum-DiLa (Formula XXVI and XXVII)

The concentration of BHB and lactate is determined in the blood samples isolated from the rats according to example 4, with the difference that DiLa (formula XXVI and XXVII) was used. Rat serum isolated every 15 minutes for a 2 hours were analyzed. The quantification was done on LC-MS/MS using isotopically labeled internal standards (7).

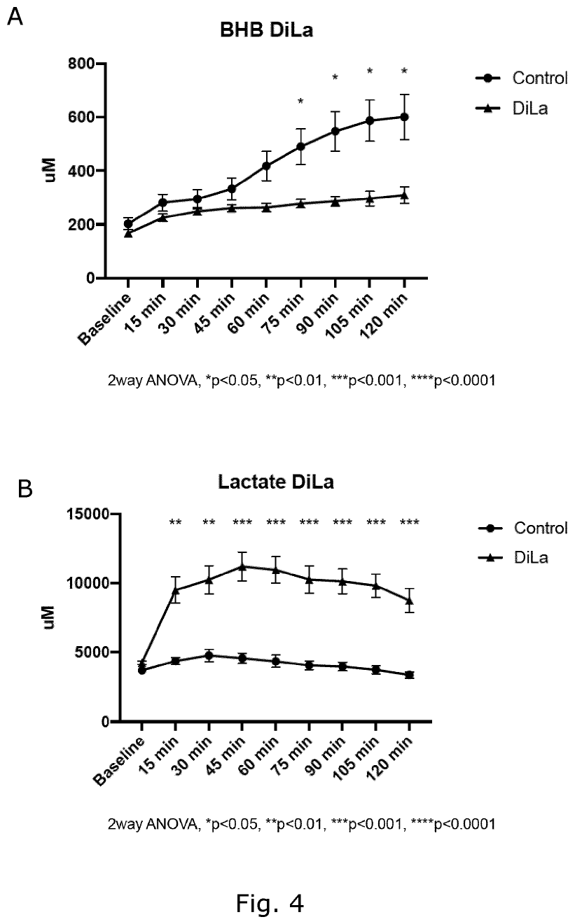

FIG. 4A: BHB concentration (UM) in rat serum isolated from two groups of rats, one treated with DiLa, one treated with the control.

FIG. 4B: Lactate concentration (UM) in rat serum isolated from two groups of rats, one treated with DiLa, one treated with the control.

### Conclusion

Here we show that treatment with DiLa (formula XXVI and XXVII) compared to the control (physiological saline 0.9%) gives rise to an increased serum concentration of lactate in rats following oral administration.

The DiLa ester appears not to release BHB directly (only lactate), but the data suggests an inhibition of BHB-mobilization relative to control during the experiment. This is compatible with a distinct inhibition of ketone formation (ketogenesis) in the liver, since levels of FFA, which are ketone body precursors, if anything were increased after DiLa ester administration.

### Example 14—Effect of the KeLa Ester (Formula XXIV and XXV) on Free Fatty Acids

The concentration of free-fatty acids (FFA) was determined in the blood samples isolated from the rats according to example 4, with the difference that KeLa (formula XXIV and XXV) was used. The level of FFA in blood reflects ongoing lipolysis and high levels of FFA induce insulin resistance; conversely agents that lower the level of free fatty acids in the blood can be used therapeutically to increase insulin sensitivity in people with type 2 diabetes and the metabolic syndrome.

Concentrations of non-esterified free fatty acids (NEFA) were measured using a NEFA-HR (2) kit (Wako, Chemicals Gmbh). Absorbance was measured by spectrometry (PHERAstar FS, BMG LABTECH, Ortenberg, Germany).

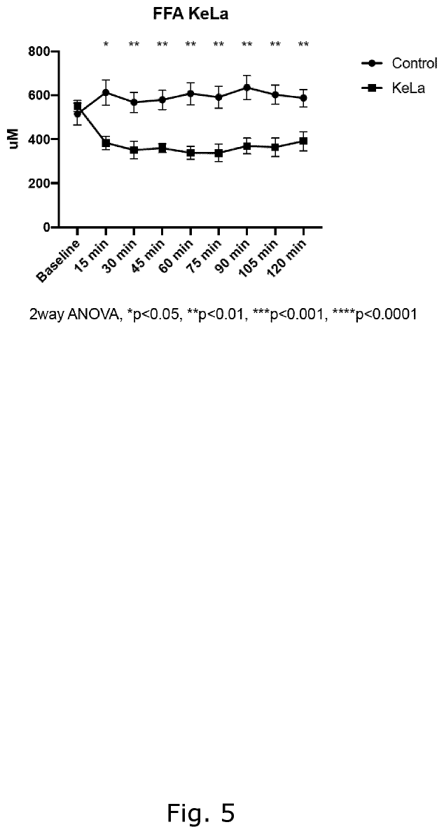

FIG. 5: The concentration (μM) of free fatty acids in the serum was strongly decreased in the rats treated with LaKe compared to rats treated with the control (physiological saline 0.9%).

### Conclusion

Here, we show that oral administration of Kela ester (formula XXIV and XXV) leads to a decreased concentration of FFA in rat serum compared to the control.

### Example 15—Effect of DiLa Ester (Formula XXVI and XXVII) on Free Fatty Acids

The concentration of free-fatty acids (FFA) was determined in the blood samples isolated from the rats according to example 4, with the difference that DiLa (formula XXVI and XXVII) was used. The level of FFA in blood reflects ongoing lipolysis and high levels of FFA induce insulin resistance; conversely agents that lower the level of free fatty acids in the blood can be used therapeutically to increase insulin sensitivity in people with type 2 diabetes and the metabolic syndrome.

Concentrations of non-esterified free fatty acids (NEFA) were measured using a NEFA-HR (2) kit (Wako, Chemicals Gmbh). Absorbance was measured by spectrometry (PHERAstar FS, BMG LABTECH, Ortenberg, Germany).

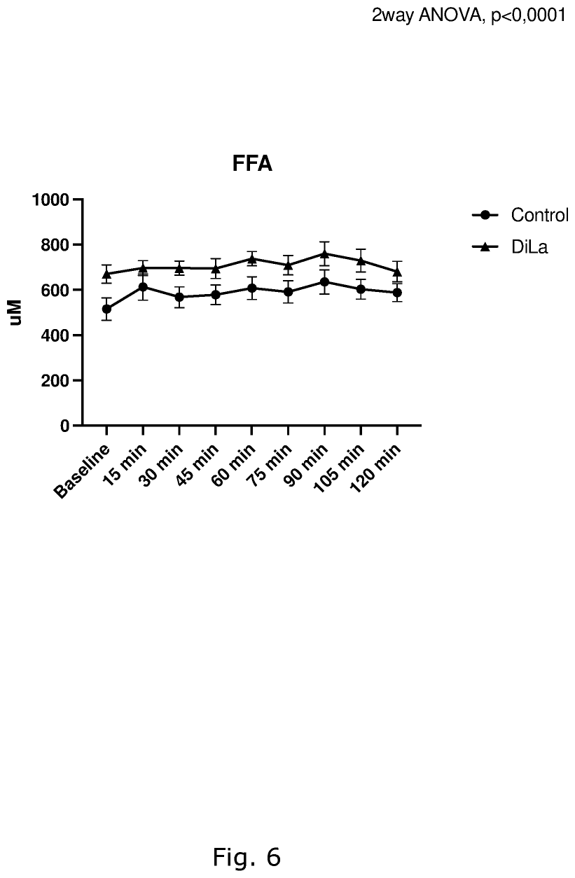

FIG. 6: The concentration (UM) of free fatty acids in the serum was not decreased in the rats treated with DiLa compared to rats treated with the control (physiological saline 0.9%).

### Conclusion

Here, we show that oral administration of DiLa ester (formula XXVI and XXVII) do not lead to a decreased concentration of FFA in rat serum compared to the control.

The presented data clearly shows that the DiLa ester results in release of lactate in large amounts (see example 8) and the compound should therefore be useful in any (medical) situation where the ability to increase lactate would be beneficial.

Further, the FFA-data for the DiLa ester, when compared to that from LaKe and KeLa, shows that the lowering of FFA is mainly driven by the BHB-components of LaKe and KeLa.

The DiLa ester may be important in the following scenarios:
when one wishes to exclusively deliver lactate.
The compound may be combined (in different ratios) with other ketone esters, such as 1,3-butanediol diacetoacetate, 1,3-butanediol dihexanoate, 1,3-butanediol, medium chain triglycerides, 3-hydroxybutyl 3-hydroxybutyrate and (3R)-hydroxybutyl (3R)-hydroxybutyrate and/KeLA and LaKe to achieve a dual delivery of BHB and lactate.

### Example 16—Chemoenzymatic Synthesis of L-(−)-Lactic Acid Esters 3 (Formula XXII) and 4 (Formula XXIII) (LaKe Esters)

### Aim of Study

To provide a Chemoenzymatic synthesis of L-(−)-lactic acid esters 3 and 4 (LaKe esters).

### Materials and Methods

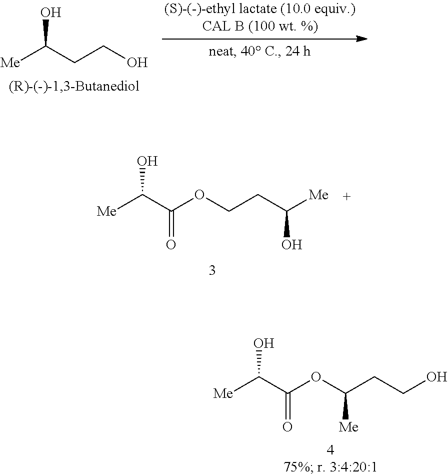 [SMILES: C[C@@H](O)CCO ; C[C@H](O)C(=O)OCC[C@@H](C)O ; C[C@H](CCO)OC(=O)[C@H](C)O]

The protocol for the enzymatic synthesis of L-(−)-lactic acid esters 3 and 4 is as follows:

(R)-(−)-1,3-Butanediol (1.50 g, 16.6 mmol, 1.0 equiv.), (S)-(−)-ethyl lactate (19.7 g, 166 mmol, 10.0 equiv.) and Cal B (1.50 g, 100 wt. %) (Lipase B Candida antarctica immobilized on Immobead 150, recombinant from yeast (≈4000 U/g), SIGMA) was placed in a 50 mL flask and heated to 40° C. The mixture was kept at this temperature and stirred carefully (300-350 rpm) for 24 h. The mixture was cooled to rt and filtered under vacuum to remove enzyme beads and the filtrate was concentrated in vacuo. The crude product was subjected to FCC (pentane/EtOAc 1:1 to 1:2) to afford the desired L-(−)-lactic acid esters (2.02 g, 75%; r. 3:4: 20:1) as a transparent oil.

### Results

Using NMR spectroscopy it was determined that the LaKe esters were produced as outlined above (data not shown)

### Conclusion

L-(−)-lactic acid esters 3 and 4 (LaKe esters) can be produced via chemoenzymatic synthesis. This provides an alternative method of preparation of the compounds which furthermore is shorter and more resource friendly.

### Example 17—Chemoenzymatic Synthesis of L-(−)-Lactic Acid Esters 7 (Formula XXVI) and 8 (Formula XXVII) (DiLa Esters)

### Aim of Study

To provide a Chemoenzymatic synthesis of L-(−)-lactic acid esters 7 and 8 (DiLa esters).

### Materials and Methods

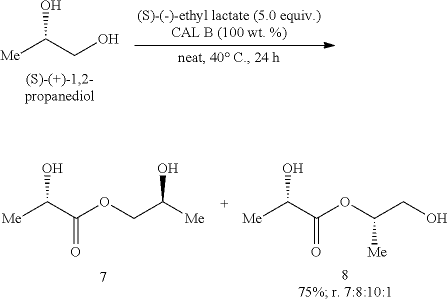 [SMILES: C[C@H](O)CO ; C[C@H](O)COC(=O)[C@H](C)O ; C[C@H](O)C(=O)O[C@@H](C)CO]

The protocol for the enzymatic synthesis of L-(−)-lactic acid esters 7 and 8 is as follows:

(S)-(+)-1,2-Propanediol (16.0 g, 0.21 mmol, 1.0 equiv.), (S)-(−)-ethyl lactate (124 g, 1.05 mmol, 5.0 equiv.) and Cal B2 (16.0 g, 100 wt. %) (Lipase B Candida antarctica immobilized on Immobead 150, recombinant from yeast (≈4000 U/g), SIGMA) was placed in a 250 mL flask and heated to 40° C. The mixture was kept at this temperature and stirred carefully (300-350 rpm) for 24 h. The mixture was cooled to rT and filtered under vacuum to remove enzyme beats and the filtrate was concentrated in vacuo. The crude product was subjected to FCC (pentane/EtOAc 1:1 to 1:2) to afford the desired L-(−)-lactic acid esters (16.1 g, 51%; r. 7:8: 10:1) as a transparent oil.

### Results

Using NMR spectroscopy it was determined that the DiLa esters were produced as outlined above (data not shown)

### Conclusion

L-(−)-lactic acid esters 7 and 8 (DiLa esters) can be produced via Chemoenzymatic synthesis. This provides an alternative method of preparation of the compounds which furthermore is shorter and more resource friendly.

Without being bound by theory, the data presented in examples 11 and 12 can also be performed for the synthesis of KeLa esters, starting from ethyl (R)-(−)-3-hydroxy butyrate and(S)-(+)-1,2-propanediol.

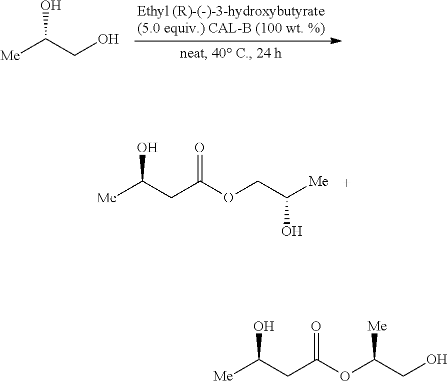 [SMILES: C[C@H](O)CO ; C[C@H](O)COC(=O)C[C@@H](C)O ; C[C@@H](O)CC(=O)O[C@@H](C)CO]

### Example 18—Synthesis of R-(−)-β-Hydroxybutyric Acid Esters 14 and 15

### (KeLa Esters) (Formula XXIV and XXV)

### Aim of Study

### Synthesis of R-(−)-β-hydroxybutyric acid esters 12 and 13 (KeLa esters)

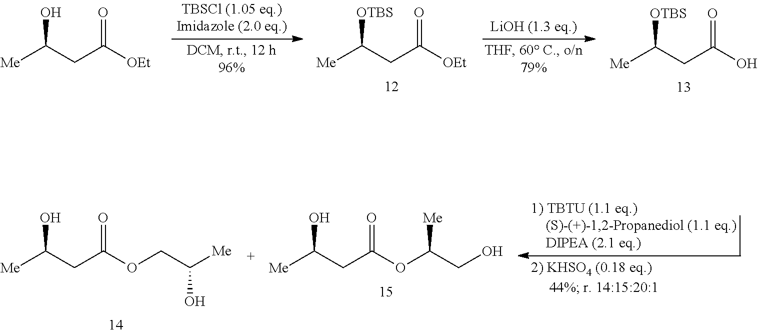 [SMILES: CCOC(=O)C[C@@H](C)O ; CCOC(=O)C[C@@H](C)O[Si](C)(C)C(C)(C)C ; C[C@H](CC(=O)O)O[Si](C)(C)C(C)(C)C ; C[C@H](O)COC(=O)C[C@@H](C)O ; C[C@@H](O)CC(=O)O[C@@H](C)CO]

### Ethyl (R)-3-((tert-butyldimethylsilyl)oxy) butanoate (12)

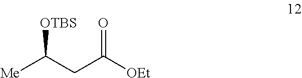 [SMILES: CCOC(=O)C[C@@H](C)O[Si](C)(C)C(C)(C)C]

To a solution of ethyl (R)-(−)-3-hydroxybutyrate (50.0 g, 378 mmol, 1.0 eq.) in anhydrous DCM (500 mL) was added TBSCl (59.8 g, 397 mmol, 1.05 eq.) followed by imidazole (51.5 g, 756 mmol, 2.0 eq.). The mixture was stirred at r.t. for 12 hours before diluted with sat. aq. NaCl and extracted with EtOAc. The organic layers were combined and washed with 5% aq. HCl and sat. aq. NaCl. The organic layer was then dried over Na₂SO₄, filtered and concentrated to afford ethyl (R)-3-(tert-butyldimethylsilyloxy) butanoate (89.0 g, 96%)

### R_{f}

0.65 (Pentane/Et₂O 95:5, KMnO₄).

### ¹H NMR

(400 MHZ, CDCl₃) δ_{H} 4.31-4.21 (m, 1H), 4.18-4.04 (m, 2H), 3.93-3.84 (m, 1H), 2.50-2.41 (m, 1H), 2.39-2.30 (m, 1H), 1.28-1.22 (t, J=7.3 Hz, 3H), 1.21-1.16 (d, J=6.22 Hz, 3H), 0.87-0.83 (s, 9H), 0.06-0.02 (d, J=8.4 Hz, 6H).

### ¹³C NMR

(101 MHZ, CDCl₃) δ_{C} 171.8, 66.0, 60.4, 45.1, 25.1, 24.1, 18.1, −4.4, −4.9.

### IR (Neat)

Uₘₐₓ/cm⁻¹ 2957, 2930, 2857, 1738, 1473, 1376, 1300, 1255, 1183, 1139, 1082.

### HRMS

[M+Na]⁺=269.1543; found: 269.1545.

[α]^{24.0}_{D}

−24.0° (C=10 mg/mL, CHCl₃).

### (R)-3-((tert-butyldimethylsilyl)oxy) butanoic acid

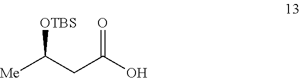 [SMILES: C[C@H](CC(=O)O)O[Si](C)(C)C(C)(C)C]

To a solution of the (R)-3-((tert-butyldimethylsilyl)oxy) butanoate (89.0 g, 361 mmol, 1.0 eq.) in THF (500 mL) at 0° C. was added a cooled aq. LiOH solution (469 mL, 1 M). The reaction mixture was stirred at 60° C. o/n before concentrated to half of the original volume and extracted with Et₂O. The organic extracts were combined before extracted with a sat. aq. NaHCO₃ solution. The aqueous layers were combined and acidified with a 1 M aq. KHSO₄ solution to reach pH≈3-4. Afterwards, the aqueous solution was extracted thourghly with Et₂O and the organic layers were combined, dried over Na₂SO₄ and concentrated to afford (R)-3-((tert-butyldimethylsilyl)oxy) butanoic acid (62.0 g, 79%) as an oil.

### R_{f}

0.21 (Pentane/EtOAc 4:1, KMnO₄).

### ¹H NMR

(400 MHZ, CDCl₃) δ_{H} 4.32-4.23 (m, 1H), 2.53-2.38 (m, 2H), 1.24-1.18 (d, J=6.2 Hz 3H), 0.88-0.84 (s, 9H), 0.08-0.04 (d, J=6.9 Hz, 6H.

### ¹³C NMR

(101 MHZ, CDCl₃) δ_{C} 177.9, 65.8, 44.6, 25.8, 23.9, 18.1, −4.4, −5.0.

### IR (Neat)

Uₘₐₓ/cm⁻¹ 3675, 2958, 2929, 2900, 2859, 1710, 1408, 1379, 1305, 1253, 1207, 1133, 1080.

### HRMS

[M]⁻=217.1265; found: 217.1262.

[α]^{24.3}_{D}

−16.5° (C=35 mg/mL, CHCl₃).

### (S)-2-Hydroxypropyl (R)-3-hydroxybutanoate (14) and(S)-1-hydroxypropan-2-yl (R)-3-hydroxybutanoate (15)

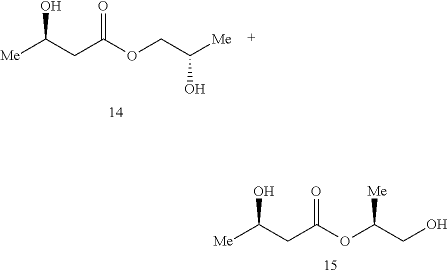 [SMILES: C[C@H](O)COC(=O)C[C@@H](C)O ; C[C@@H](O)CC(=O)O[C@@H](C)CO]

The acid (62 g, 284 mmol, 1.0 eq.), TBTU (101 g, 312 mmol, 1.1 eq.) and DIPEA (104 mL, 596 mmol, 2.1 eq.) was dissolved in anhydrous DMF (700 mL) and the mixture was stirred at r.t. for 1 hour. (S)-(+)-1,2-propanediol (23.5 mL, 312 mmol, 1.1 eq.) in anhydrous DMF (30 mL) was then added and the reaction mixture was stirred o/n. at ambient temperature. The reaction mixture was diluted with CH₂Cl₂ and the resulting mixture was washed with 1 M aq. HCl, aq. NaHCO₃ and water sequentially. The organic layers were then combined, dried over Na₂SO₄, filtered and concentrated to afford the crude product.

The crude TBS-ether was dissolved in THF:H₂O:MeOH (2.5:2.5:1, 500 mL) was added KHSO₄ (7.14 g, 52.5 mmol, 0.18 eq.) and the mixture was stirred at r.t. o/n. MeOH was removed under reduced pressure and water was added before the aqueous layer was washed with Et₂O. Afterwards, the aqueous phase was extracted thourghly with EtOAc (20×). The organic layers were collected and dried over Na₂SO₄, filtered and concentrated. The crude product was purified with FCC (Pentane/EtOAc=1:1 to 1:2) to afford the desired products (19.7 g, 44%; r. 14:15: 20:1).

### R_{f}

0.28 (Pentane/EtOAc 1:4, CAM).

### ¹H NMR

14: (400 MHZ, CDCl₃) δ_{H} 4.25-4.14 (m, 1H), 4.13-4.07 (dd, J=10.7 Hz, 2.6 Hz, 1H), 4.05-3.97 (m, 1H), 3.97-3.90 (dd, J=10.7 Hz, 7.5 Hz, 1H), 3.57-4.34 (s, 1H), 3.23-3.11 (s, 1H), 2.53-2.38 (m, 2H), 1.24-1.20 (d, J=6.3 Hz, 3H), 1.19-1.15 (d, J=6.3 Hz, 3H).

### ¹³C NMR

14: (101 MHZ, CDCl₃) δ_{C} 172.7, 69.7, 65.8, 64.6, 43.3, 22.8, 19.1.

### IR (Neat)

Uₘₐₓ/cm⁻¹ 3368, 2974, 2934, 1716, 1377, 1291, 1178, 1123, 1072.

### HRMS

[M+Na]⁺=185.0784; found: 185.0788.

[α]^{24.3}_{D}

−17.4° (C=35 mg/mL, CHCl₃).

### Example 19—Chemical Synthesis of diLaKe

### Aim

To provide a chemical synthesis of diLaKe.

### Materials and Methods

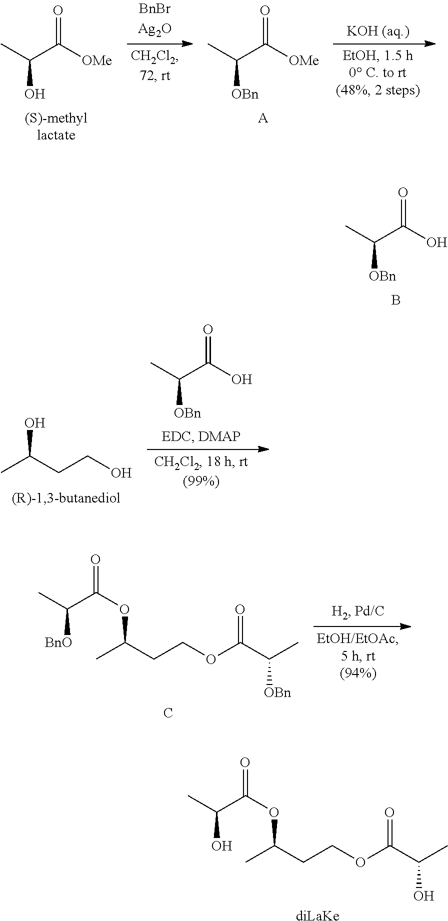

### Methyl(S)-2-(benzyloxy) propanoate (B)

To a solution of(S)-methyl lactate (225 mmol, 21.5 mL) in anhydrous dichloromethane (425 mL) was added benzyl bromide (281 mmol, 33.6 mL) and silver (I) oxide (281 mmol, 65.2 g). The reaction mixture was stirred for 72 hours at room temperature under an argon atmosphere. Then, the reaction mixture was filtered through a pad of Celite® and the filtrate was evaporated under reduced pressure (200 mbar) to afford crude A (43.15 g) as a colourless oil which was used in the next step without further purification.

1M KOH aqueous solution (225 mmol, 225 mL) was slowly added to a cooled solution of crude A (43.15 g) in ethanol (225 mL) and the reaction mixture was stirred for 30 minutes at 0° C. and then for 1 hour at room temperature. Then, the reaction mixture was concentrated to half of the volume at reduced pressure and the resulting aqueous phase was washed with diethyl ether (2×200 mL). The aqueous phase was acidified (pH ca. 2) with concentrated HCl aqueous solution and then extracted with dichloromethane (2×200 mL). The combined organic layers were dried with anhydrous Na₂SO₄, filtered and evaporated under reduced pressure to afford B (19.65 g, 48% yield, 2 steps) as a colourless oil that finally crystallises as a white solid. The obtained 1H-NMR data matched those reported in the bibliography (J. Nat. Prod. 2015, 78, 476-485).

R_{f} 0.48, tailed (Et₂O/hexane 7:3, UV and KMnO₄). ¹H-NMR (400 MHZ, CDCl₃, δ): 7.42-7.28 (m, 5H), 4.70 (d, 1H; J=11.4), 4.55 (d, 1H, J=11.4), 4.12 (q, 1H, J=6.8), 1.50 (d, 3H, J=6.8).

### (R)-butane-1,3-diyl (2S,2'S)-bis(2-(benzyloxy) propanoate) (C)

To a solution of(S)-2-benzyloxypropanoic acid B (109 mmol, 19.62 g) in anhydrous dichloromethane (340 mL) was sequentially added EDC hydrochloride (145 mmol, 27.84 g), triethylamine (145 mmol, 20.2 mL), DMAP (36.3 mmol, 4.44 g) and a solution of (3R)-butane-1,3-diol (36.3 mmol, 3.27 g) in anhydrous dichloromethane (20 mL). The reaction mixture was stirred at rt for 20 hours under an argon atmosphere. Then, the reaction mixture was washed with 1M HCl aqueous solution (400 mL) and then with saturated NaHCO₃ aqueous solution (400 mL). The organic phase was dried with anhydrous Na₂SO₄, filtered and evaporated under reduced pressure. The resulting residue was purified by column chromatography (eluent 1: hexane/AcOEt 9:1, eluent 2: hexane/AcOEt 7:3) to afford C (14.90 g, 99% yield) as a colourless oil.

R_{f} 0.68 (Et₂O/hexane 1:1, UV and KMnO₄). ¹H-NMR (400 MHZ, CDCl₃, δ): 7.44-7.27 (m, 10H), 5.18-5.06 (m, 1H), 4.69 (d, 1H, J=11.6), 4.69 (d, 1H, J=11.6), 4.45 (d, 1H, J=11.6), 4.44 (d, 1H, J=11.6), 4.30-4.21 (m, 1H), 4.21-4.13 (m, 1H), 4.07 (q, 1H, J=6.8), 4.04 (q, 1H, J=6.8), 2.05-1.86 (m, 2H), 1.45 (d, 3H, J=6.8), 1.43 (d, 3H, J=6.8), 1.30 (d, 3H, J=6.4). ¹³C-NMR (100 MHZ, CDCl₃, δ): 173.3, 172.9, 137.7, 128.6, 128.6, 128.1, 128.1, 128.0, 128.0, 74.1, 74.0, 72.2, 72.1, 68.2, 61.0, 35.0, 20.2, 18.9, 18.9. IR (neat, vₘₐₓ/cm⁻¹): 1743, 1140, 1116. [α]_{D}²⁶ −101.8 (c=1.0, CHCl₃). HRMS (ESI⁺): m/z [M+Na]⁺ calculated for C₂₄H₃₀NaO₆ 437.1935, found 437.1948.

### (R)-butane-1,3-diyl (2S,2'S)-bis(2-hydroxypropanoate) (diLaKe)

To a solution of compound C (35.9 mmol, 14.88 g) in EtOH/AcOEt 1:1 (500 mL) was added palladium on carbon (1.48 g, 10% Pd/C) and the reaction mixture was stirred at room temperature for 5 hours under an atmosphere of hydrogen. Then, the reaction mixture was filtered through a pad of Celite® (washing with dichloromethane) and the filtrate was evaporated under reduced pressure. The resulting residue was filtered through a pad of silica gel (eluent 1: Et₂O/hexane 1:1, eluent 2: Et₂O) to afford diLaKe (7.90 g, 94% yield) as a colourless oil. R_{f} 0.13 (Et₂O/hexane 4:1, KMnO₄). ¹H-NMR (400 MHZ, CDCl₃, δ): 5.17-5.04 (m, 1H), 4.33-4.15 (m, 4H), 2.91-2.80 (m, 2H), 2.06-1.88 (m, 2H), 1.41 (d, 3H, J=6.8), 1.40 (d, 3H, J=7.2), 1.30 (d, 3H, J=6.0). ¹³C-NMR (100 MHZ, CDCl₃, δ): 175.7, 175.4, 69.3, 66.9, 66.8, 61.7, 34.8, 20.5, 20.4, 20.1. IR (neat, vₘₐₓ/cm⁻¹): 3442 (br.), 1729, 1123. [α]_{D}²³ −29.8 (c=1.0, CHCl₃). HRMS (ESI⁺): m/z [M+Na]⁺ calculated for C₁₀H₁₈NaO₆ 257.0996, found 257.1004.

### Results

Using NMR spectroscopy and HRMS, it has been determined that diLaKe is produced as outlined above.

### Conclusion

diLaKe has been produced via chemical synthesis.

### Example 20—Chemoenzymatic Synthesis of diLaKe (Formula II)

### Aim

To provide an alternative chemoenzymatic synthesis of diLaKe.

### Materials and Methods

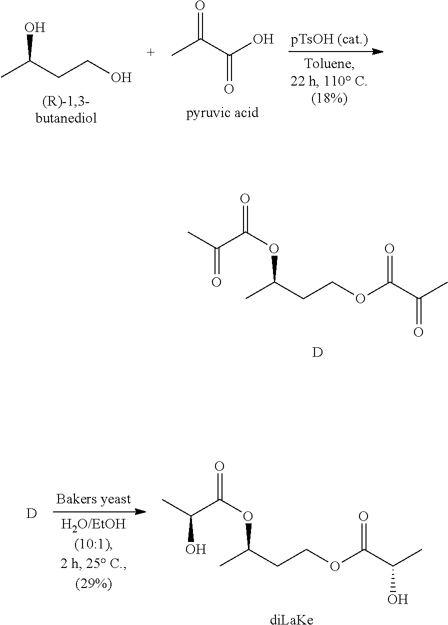

### (R)-butane-1,3-diyl bis(2-oxopropanoate) (D)

To a mixture of pyruvic acid (400 mmol, 35.2 g) and (R)-1,3-butanediol (100 mmol, 9.0 g) in anhydrous toluene (700 mL) was added p-toluenesulfonic acid monohydrate (6 mmol, 1.1 g). The reaction mixture was stirred for 22 hours at reflux with azeotropic removal of water (Dean-Stark). Then, the reaction mixture was cooled to room temperature. The solvent was evaporated under reduced pressure and the resulting residue was dissolved in dichloromethane (500 mL) and washed with saturated NaHCO₃ aqueous solution (2×500 mL). The organic layer was dried with anhydrous Na₂SO₄, filtered and evaporated under reduced pressure. The resulting residue was purified by column chromatography (AFCC; eluent 1: heptane/AcOEt 4:1, eluent 2: heptane/AcOEt 4:1 to 3:2) to afford D (4.22 g, 18% yield) as a yellowish oil.

R_{f} 0.48 (Et₂O/hexane 4:1, KMnO₄). ¹H-NMR (400 MHZ, CDCl₃, δ): 5.21-5.10 (m, 1H), 4.37 (ddd, 1H, J=11.2, J=7.6, J=5.2), 4.28 (ddd, 1H, J=11.2, J=6.0, J=6.0), 2.47 (s, 3H), 2.47 (s, 3H), 2.20-2.00 (m, 2H), 1.38 (d, 3H, J=6.4). ¹³C-NMR (100 MHZ, CDCl₃, δ): 191.9, 191.5, 160.8, 160.5, 70.7, 62.6, 34.5, 26.8, 26.7, 20.0. IR (neat, vₘₐₓ/cm⁻¹): 1729, 1143. [α]_{D}²² −24.6 (c=1.0, CHCl₃). HRMS (ESI⁺): m/z [M+Na]⁺ calculated for C₁₀H₁₄NaO₆ 253.0683, found 253.0683.

### (R)-butane-1,3-diyl (2S,2'S)-bis(2-hydroxypropanoate) (diLaKe)

A suspension of Baker's yeast (YSC2, Sigma-Aldrich, 4.60 g) in distilled water (46 mL) was stirred for 0.5 hours at 25° C. A solution of compound D (1.00 mmol, 230 mg) in ethanol (4.6 mL) was then added and the resulting suspension was stirred for 2 hours at 25° C. Then, dichloromethane (50 mL) was added to the reaction mixture, and the whole mixture was vigorously stirred for 30 minutes. After adding some Celite®, the reaction mixture was vacuum-filtered through filter paper and the phases were separated. The aqueous phase was extracted with dichloromethane (50 mL) and the combined organic layers were dried with anhydrous Na₂SO₄, filtered and evaporated under reduced pressure. The resulting residue was purified by column chromatography (AFCC; eluent 1: heptane/AcOEt 3:2, eluent 2: heptane/AcOEt 3:2 to 3:7) to afford diLaKe (68 mg, 29% yield) as a colourless oil.

The obtained ¹H-NMR and ¹³C-NMR data and HPLC-traces matched those obtained for diLaKe obtained via chemical synthesis.

### Results

Using NMR spectroscopy and HRMS, it has been determined that diLaKe is produced as outlined above.

### Conclusion

diLaKe has been produced via an alternative chemoenzymatic synthesis.

### Example 21—Animal Experiments and Quantification of BHB and Lactate in Rat Serum-diLaKe (Formula II)

### Materials and Methods

As provided in examples 3 and 4 with the difference that values were compared to baseline (internal control). Furthermore sampling was conducted until 180 minutes.

### Results

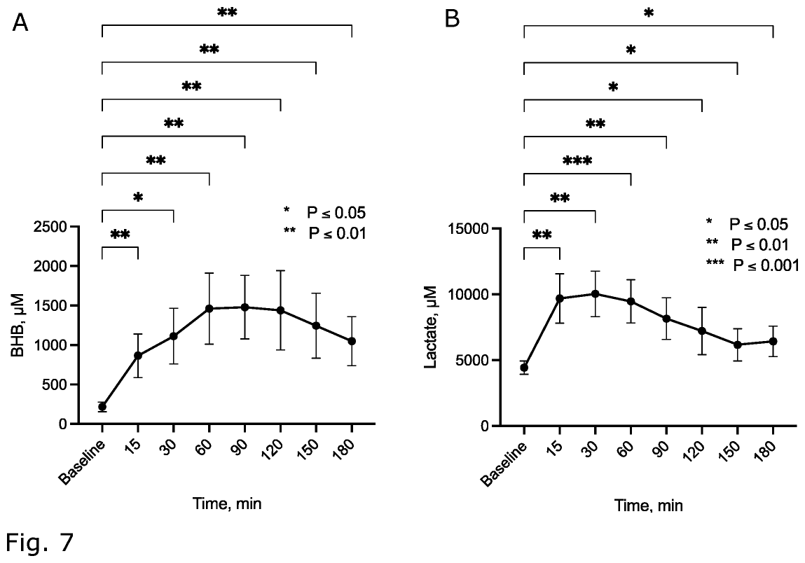

As seen by FIG. 7 diLaKe results in an increase in serum levels of BHB and Lactate. As expected, the increased serum concentrations of both BHB and lactate decrease again over time.

### Conclusion

Here we show that treatment with diLaKe, gives rise to an increased serum concentration of both BHB and lactate in rats following oral administration.

### Example 22—Animal Experiments and Effect of the diLaKe Ester (Formula II) on Free Fatty Acids

### Materials and Methods

As provided in examples 3 and 5 with the difference that values were compared to baseline (internal control). Furthermore, sampling was conducted until 180 minutes.

### Results

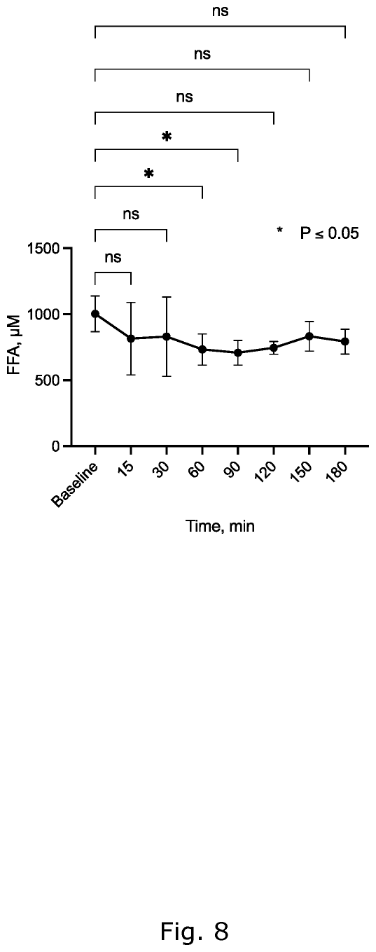

As seen in FIG. 8 diLaKe result in a decrease in FFA-levels. As expected, the decreased levels of FFA increase again over time.

### Conclusion

Here, we show that oral administration of diLaKe leads to a decreased concentration of FFA in rat serum compared to the control.

### Example 23—Effect of the diLaKe Ester (Formula II) on N-Lactoyl-Phenylalanine (Lac-Phe)

### Aim

To demonstrate that N-lactoyl-phenylalanine (Lac-Phe) increases in concentration following oral ingestion of diLaKe.

### Materials and Methods

As provided in example 21 with the difference that N-lactoyl-phenylalanine (Lac-Phe) was quantified using LC-MS/MS analyses.

LC-MS/MS analyses were carried out as follows: Forty μL rat plasma was diluted with 100 μL water in a 2 mL tube, added 115 μL methanol (Merck hypergrade) and 420 μL acetonitrile (Merck hypergrade), vortex mixed, incubated for 5 min at room temperature, and then centrifuged at 10,000×g for 5 min. Five hundred microliter supernatant was transferred to a 96-well plate (Eppendorf 1 mL deepwell), evaporated to dryness under a nitrogen gas flow at 30° C., and then reconstituted in 100 μL water with 0.1% formic acid.

Pure calibrant samples were prepared from an authentic standard compound (N,L-lactoyl phenylalanine), using the plasma sample preparation method at concentrations equivalent to 0, 1, 5, 10, 50, 100, 500, and 1000 UM in the original sample.

A 7 μL sample volume was injected to an ultra high performance liquid chromatography system (Waters Acquity UPLC) coupled to a triple-quadrupole mass spectrometer (Waters Xevo TOS) with a T3-UPLC column (Waters Acquity UPLC BEH T3, 1.8 μm, 100×2.1 mm) maintained at 40° C. The flow rate was 0.35 mL/min and the separation was initiated with 100% mobile phase A (water with 0.1% formic acid) for 2 min, and then changed through linear gradients to 40% B (methanol:acetonitrile 1;1 with 0.1% formic acid) (2-6 min), to 60% B (6-7) min, and to 88% B (7-8 min) and to 100% B (9-10 min). A linear gradient back to 100% A at 11 min was maintained for 3 minutes for column equilibration resulting in a total runtime of 14 minutes.

The sample was introduced to the mass spectrometer through electrospray ionization in the negative mode with a capillary voltage of 2.2 kV and the source and desolvation temperatures were 150 and 500° C., and the nitrogen gas flows were 50 L/h (cone) and 800 L/h (desolvation). The analytes were detected in the multiple reaction monitoring mode with the following transitions (m/z) and cone voltage/collision energies (CV/CE), L-Lac-Phe (quantification ion m/z: 236.2>88.1, CV/CE: 25V/15 eV and qualifier ion m/z: 236.2>147.1, CV/CE: 25V/15 eV). The data acquisition and processing including peak integration were performed using MassLynx 4.2 (Waters).

Calibration curves were constructed by linear regression of the peak area of the pure calibrant samples versus the nominal analyte concentrations and based on eight points. The Lac-Phe concentrations in rat plasma samples were derived from their peak area with reference to the calibration curve. Values at the different timepoints were compared to baseline (internal control).

### Results

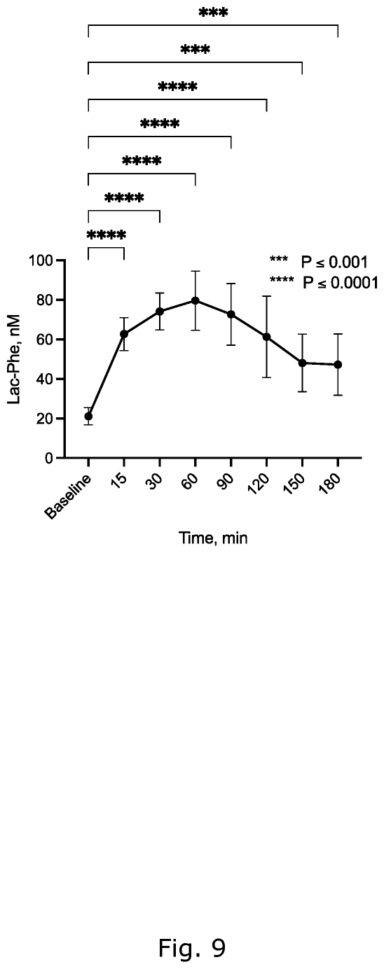

As can be seen in FIG. 9 we have shown that treatment with diLaKe (formula II), gives rise to an increased serum concentration of Lac-Phe in rats following oral administration. As expected, the increased levels of Lac-Phe decrease again over time as the high levels of lactate also decrease.

### Conclusion

Oral administration of diLaKe (formula II) gives rise to increased serum concentrations of Lac-Phe.

### Example 24—Synthesis of (R)-butane-1,3-diyl bis(2-oxopropanoate) (diPyKe) (formula LXII)

### Aim

To provide a chemical synthesis of diPyKe.

### Materials and Methods

The synthesis of diPyKe is described above in example 20, as compound D.

### Results

As presented in example 20, the chemoenzymatic synthesis of diLaKe is firstly driven by the synthesis of the intermediate (R)-butane-1,3-diyl bis(2-oxopropanoate) (compound D) also referred to herein as formula LXII or diPyKe. Surprisingly, the reaction can be stopped at this intermediate, which is stable on its own.

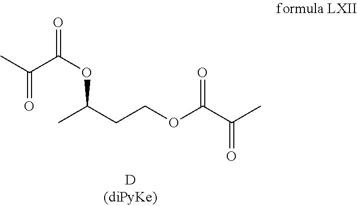 [SMILES: CC(=O)C(=O)OCC[C@@H](C)OC(=O)C(C)=O]

### Conclusion

We were able to obtain diPyKe, a novel compound suitable for oral administration.

### Example 25—Quantification of BHB and Lactate in Rat Serum-diPyKe (Formula LXII)

### Materials and Methods

As provided in examples 3 and 4 with the difference that values were compared to baseline (internal control). Furthermore sampling was conducted until 180 minutes and the experiment was carried out with a single rat.

### Results

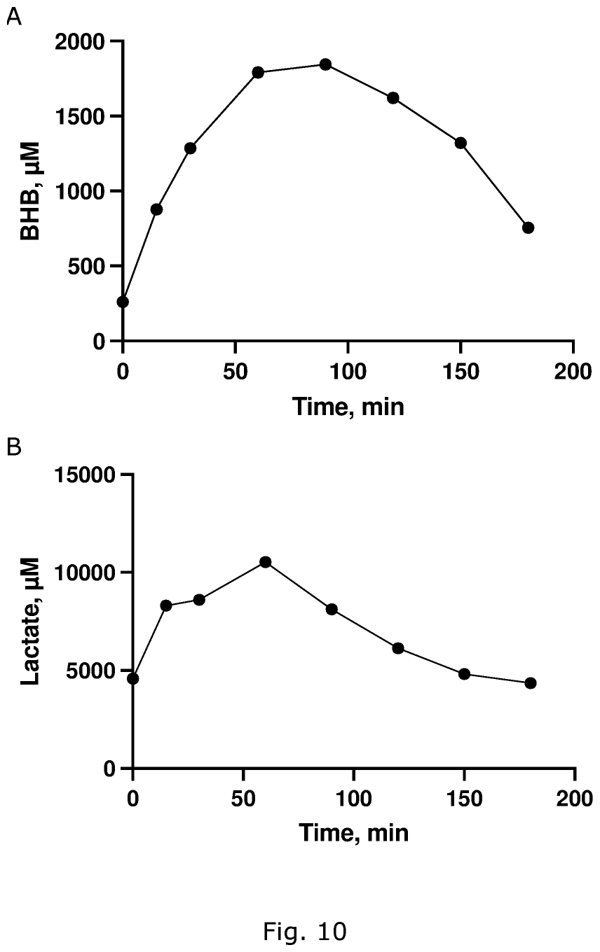

As seen by FIG. 10 diPyKe results in an increase in serum levels of BHB and lactate. As expected, the increased serum concentrations of both BHB and lactate decrease again over time.

### Conclusion

Here we show that treatment with diPyKe, gives rise to an increased serum concentration of both BHB and lactate in a rat following oral administration.

### Example 26—Effect of the diPyKe ester (formula LXII) on N-lactoyl-phenylalanine (Lac-Phe)

### Aim

To demonstrate that N-lactoyl-phenylalanine (Lac-Phe) increases in concentration following oral ingestion of diPyKe.

### Materials and Methods

As provided in example 23 with the difference that the experiment was carried out with a single rat.

### Results

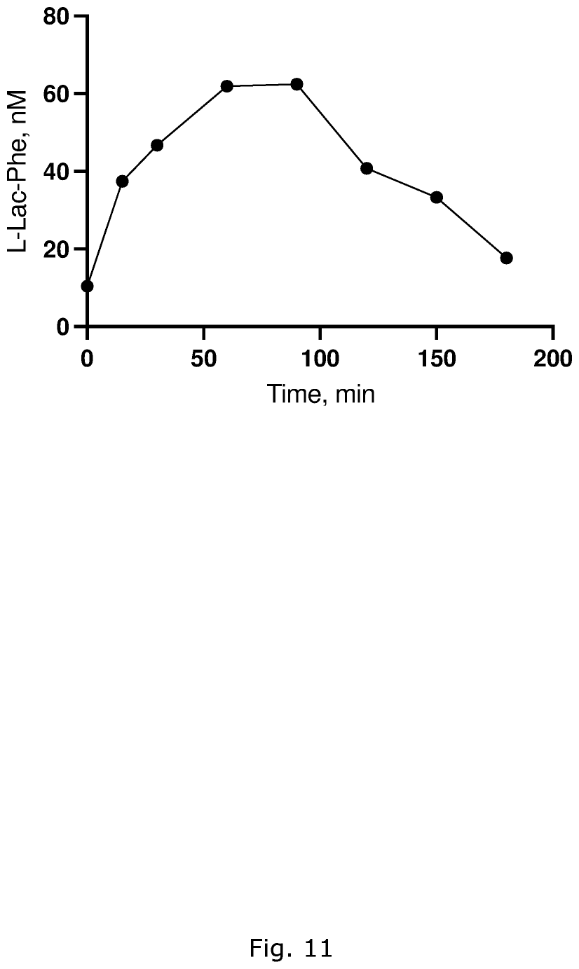

As can be seen in FIG. 11 we have shown that treatment with diPyKe (formula II), gives rise to an increased serum concentration of Lac-Phe in a rat following oral administration. As expected, the increased levels of Lac-Phe decrease again over time as the high levels of lactate also decrease.

### Conclusion

Oral administration of diPyKe (formula LXII) gives rise to increased serum concentrations of Lac-Phe.

### REFERENCES

1—Sun, S. Li, H., Chen, J., Qian, Q. Lactic Acid: No Longer an Inert and End-Product of Glycolysis. Physiology, 2017, 32, 453-463. doi: 10.1152/physiol.00016.2017.
2—Puchalska, P., Crawford, P. A. Multi-dimensional Roles of Ketone Bodies in Fuel Metabolism, Signaling, and Therapeutics. Cell Metab. 2017 25, 262-284. doi: 1016/j.cmet.2016.12.022.
3—Nielsen, R., Møller, N., Gormsen, L, C., Tolbod, L. P., Hansson, N. H., Sorensen, J., Harms, H. J., Frøkiær, J., Eiskjaer, H., Jespersen, N. R., Mellemkjaer, S., Lassen, T. R., Pryds, K., Bøker, H. E., Wiggers, H. Cardiovascular Effects of Treatment With the Ketone Body 3-Hydroxybutyrate in Chronic Heart Failure Patients. Circulation. 2019, 139, 2129-2141. doi: 10.1161/CIRCULATIONAHA.118.036459.
4—Thomsen, H. H., Rittig, N., Johannsen, M., Møller, A. B., Jørgensen, J. O., Jessen, N., Møller, N. Effects of 3-hydroxybutyrate and free fatty acids on muscle protein kinetics and signaling during LPS-induced inflammation in humans: anticatabolic impact of ketone bodies. Am. J. Clin. Nutr. 2018, 108, 857-867. doi: 10.1093/ajcn/nqy170
5—Lauritsen, K. M., Søndergaard, E., Svart, M., Møller, N., Gormsen, L. C. Ketone Body Infusion Increases Circulating Erythropoietin and Bone Marrow Glucose Uptake. Diabetes Care. 2018, 41, e152-e154. doi: 10.2337/dc18-1421.
6—M. C. Petersen, G. I. Shulman, Mechanisms of Insulin Action and Insulin Resistance. Physiol Rev. 98, 2133-2223 (2018).
7—Lambert K. Sørensen, Nikolaj F. Rittig, Emil F. Holmquist, Karl Anker Jørgensen, Jens Otto Lunde Jørgensen, Niels Møller, Mogens Johannsen, Simultaneous determination of β-hydroxybutyrate and β-hydroxy-β-methylbutyrate in human whole blood using hydrophilic interaction liquid chromatography electrospray tandem mass spectrometry, Clinical Biochemistry, Volume 46, Issue 18, 2013, Pages 1877-1883.
8—Rabinowitz & Sven Enerbäck. Lactate: the ugly duckling of energy metabolism. Nature Metabolism volume 2, pages 566-571 (2020).
9—Brooks, G. A., Arevalo, J. A., Osmond, A. D., Leija, R. G., Curl, C. C. and Tovar., A. P. Lactate in contemporary biology: A phoenix risen. J. Physiol. 1-23 (2021).
10—Mayer, S. C.; Ramanjulu, J; Vera, M. D.; Pfizenmayer, A. J; Joullié, M. M, J. Org. Chem., 1994, 59, 5192-5205

1. A compound selected from the group consisting of: 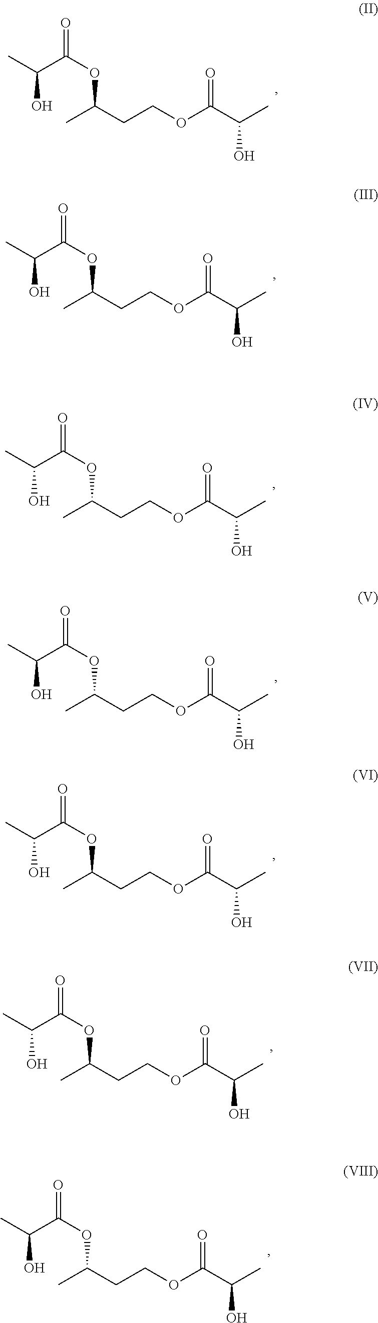 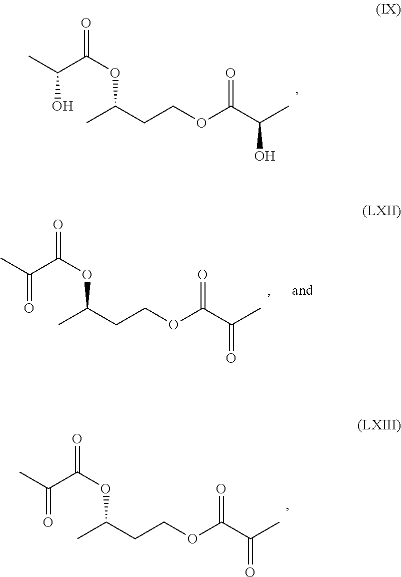
or any pharmaceutically acceptable salts thereof.

2-23. (canceled)

24. A composition comprising one or more compounds selected from the group consisting of: 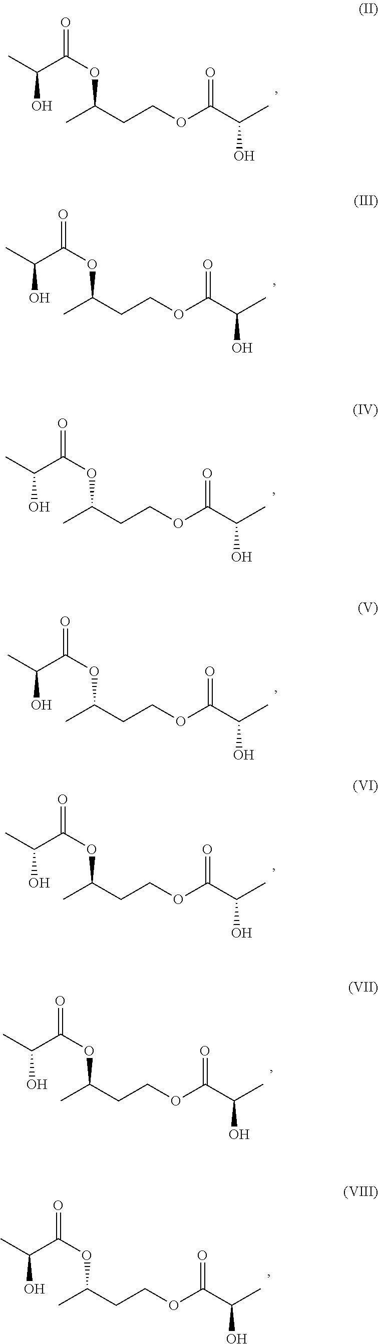 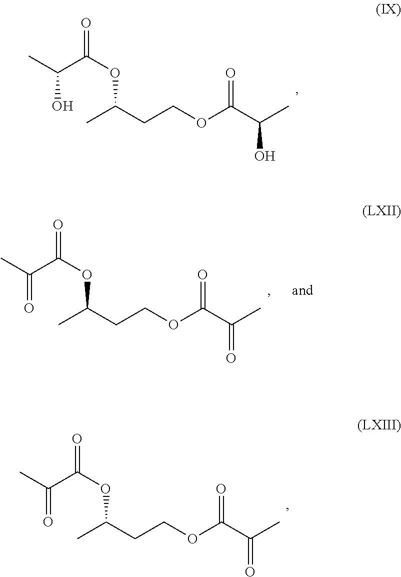
or any pharmaceutically acceptable salts thereof.

25. The composition according to claim 24, comprising a compound according to formula II and/or an isoform thereof.

26. The composition according to claim 24, comprising a compound according to formula LXII and/or an isoform thereof.

27. The composition according to claim 24, further comprising one or more compounds selected from the group consisting of:
a compound with the formula X
a) 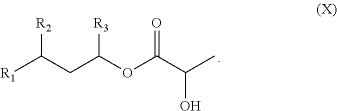
a compound of the formula XI 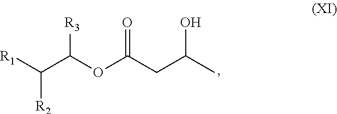
and
a compound of formula XII 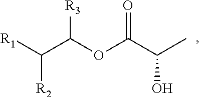
or any pharmaceutically acceptable salts thereof,
wherein R₁, R₂ and R₃ are CH₃, OH and H respectively or R₁, R₂ and R₃ are OH, H and CH₃ respectively.

28. The composition according to claim 24, further comprising one or more compounds selected from the group consisting of:
a compound with formula XXII 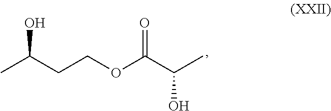
a compound of the formula XXXXI 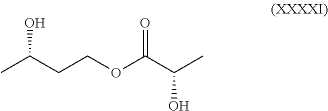
a compound with formula XXIII 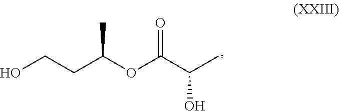
a compound of the formula XXXXII 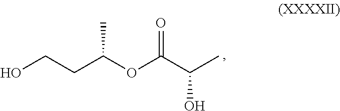
a compound of the formula XXXXIII 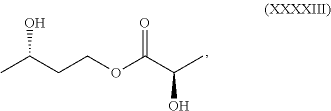
a compound of the formula XXXXIV 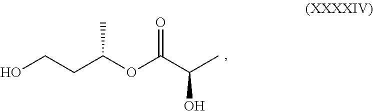
a compound with formula XXIV 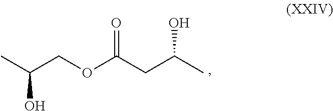
a compound with formula XXV 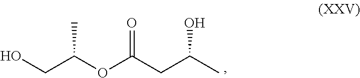
a compound with formula XXVI 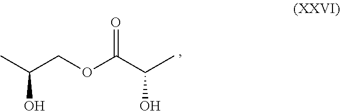
a compound of the formula XXXXV 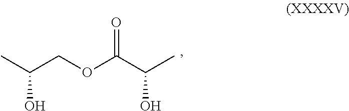
a compound with formula XXVII 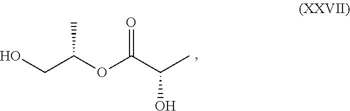
a compound of the formula XXXXVI 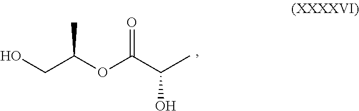
a compound of the formula XXXXVII 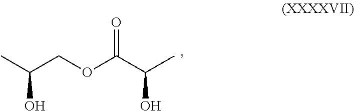
a compound of the formula XXXXVIII 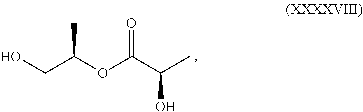
a compound of the formula XXXXIX 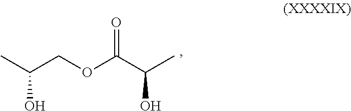
a compound of the formula L, 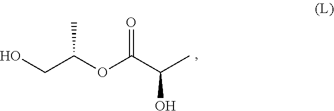
a compound with formula XXVIII 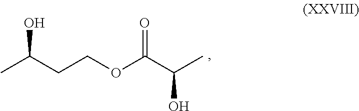
and
a compound with formula XXIX 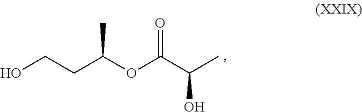
or any pharmaceutically acceptable salts thereof.

29. The composition according to claim 24, comprising a compound of formula II and/or an isoform thereof and at least one compound selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.

30. The composition according to claim 24, comprising a compound of formula LXII and/or an isoform thereof and at least one compound selected from the group consisting of 1,3-butanediol diacetoacetate, 1,3-butanediol dihexanoate, 1,3-butanediol, medium chain triglycerides, medium chain fatty acids, 3-hydroxybutyl 3-hydroxybutyrate and (3R)-hydroxybutyl (3R)-hydroxybutyrate.

31. The composition according to claim 24, wherein the composition comprises a compound of formula II and/or an isoform thereof and a compound of formula LXII and/or an isoform thereof.

32. The compound according to claim 1 comprised in a pharmaceutical composition.

33. A method of:
treating a condition, which is caused by, exacerbated by or associated with elevated plasma levels of free fatty acids in a human or animal subject;
treating a condition where weight loss or weight gain is implicated;
suppressing appetite, treating obesity, promoting weight loss, maintaining a healthy weight or decreasing the ratio of fat to lean muscle;
preventing or treating a condition selected from cognitive dysfunction, a neurodegenerative disease or disorder, muscle impairment, fatigue and muscle fatigue;
treating a patient suffering from a condition selected from diabetes, hyperthyroidism, metabolic syndrome X, or for treating a geriatric patient;
treating, preventing, or reducing the effects of, neurodegeneration, free radical toxicity, hypoxic conditions or hyperglycemia;
preventing or treating a neurodegenerative disease or disorder selected from Alzheimer's disease, Parkinson's disease, amyotrophic lateral sclerosis, epilepsy, astrocytoma, glioblastoma and Huntington's chorea;
promoting alertness or improving cognitive function in a subject;
non-therapeutic treatment of muscle impairment or fatigue;
non-therapeutically increasing serum concentration of Lac-Phe;
non-therapeutic treatment of cardiac muscle fatigue or skeletal muscle fatigue; or
non-therapeutic treatment of heart failure;
wherein the method comprises administering to a subject in need thereof the compound according to claim 1.

34. The method according to claim 33, wherein the compound or compound is administered by a method selected from the group consisting of oral administration, nasal administration, intradermal injection, intravenous administration, intramuscular administration, subcutaneous administration, intratracheal administration and transdermal administration.

35. The method according to claim 33, wherein the compound or composition is administered in a dose of 0.05-1.5 g/kg;
or
being administered in a dose of 0.05-15 g/kg;
or
being administered in a daily dosage in the range 0.15-45 g/kg.

36. The method according to claim 33, wherein the compound or compound is administered to a subject being selected from the group consisting of humans of all ages, mammals, including cattle, pigs, horses, sheep, goats, mink, ferrets, hamsters, cats, dogs; and/or birds, preferably the subject is a human.

37. The compound according to claim 1 comprised in a food ingredient or a food product.

38. The compound according to claim 1 comprised in a food product, wherein the food is selected from the group consisting of a nutraceutical, a food supplement, a dietary supplement, a feed, bar, sugar bar, protein bar, powder, gel, beverage, drink, yoghurt, chewing gum, dairy product, sports drink, confectionary product, ice cream, capsule, tablet, sachet, and pouch.

39. A process for producing a compound, the process comprising:
a) providing 1,3 butanediol, and pyruvic acid;
b) reacting said compounds from step a) in the presence of a catalytic acid; and
c) obtaining a compound of formula LXII 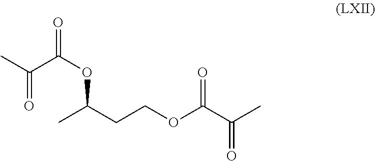 [SMILES: CC(=O)C(=O)OCC[C@@H](C)OC(=O)C(C)=O]
and/or an isoform thereof.

40. The process according to claim 39, wherein the process further comprises the steps d), and e):
d) reacting the compound of formula LXII of step c) in the presence of a dehydrogenase; and
e) obtaining a compound of formula II 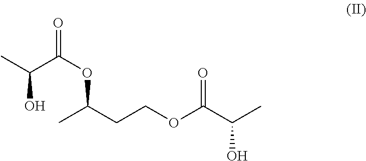 [SMILES: C[C@H](CCOC(=O)[C@H](C)O)OC(=O)[C@H](C)O]
and/or an isoform thereof.
